SEQUENCE LISTING (99 sequences)

SEQ ID NO: 1 - protein, 174 aa, Arabidopsis thaliana
MVSLLKLQKQHRTLNPYSLRKRKKEMADHQQHQQQQQPIMRSLHESSPSTRQIVRFVTAATIGLSLLVLSGLTLTGTVIGLIVATPLMVLFSPVLVPAVITIGLLTMGFLFSGGCGVAAATALTWIYKYVTGKHPMGADKVDYARMRIAEKAKELGHYTHSQPQQTHQTTTTTH

SEQ ID NO: 2 - protein, 199 aa, Arabidopsis thaliana
MADTHRVDRTDRHFQFQSPYEGGRGQGQYEGDRGYGGGGYKSMMPESGPSSTQVLSLLIGVPVVGSLLALAGLLLAGSVIGLMVALPLFLLFSPVIVPAALTIGLAMTGFLASGMFGLTGLSSISWVMNYLRGTRRTVPEQLEYAKRRMADAVGYAGQKGKEMGQHVQNKAQDVKQYDISKPHDTTTKGHETQGRTTAA

SEQ ID NO: 3 - protein, 174 aa, Arabidopsis thaliana; identical to SEQ ID NO: 1

SEQ ID NO: 4 - protein, 183 aa, Arabidopsis thaliana
MADVRTHSHQLQVHPQRQHEGGIKVLYPQSGPSSTQVLAVFVGVPIGGTLLTIAGLTLAGSVIGLMLAFPLFLIFSPVIVPAAFVIGLAMTGFLASGAIGLTGLSSMSWVLNYIRRAGQHIPEELEEAKHRLADMAEYVGQRTKDAGQTIEDKAHDVREAKTFDVRDRDTTKGTHNVRDTKTT

SEQ ID NO: 5 - protein, 169 aa, Arabidopsis thaliana
MADRTNPSSHTQQRPIYNSTTVPRSNTTTNHPLSSLLRQLLQSQSPNHSGQLFGFLAFFISGGILLLLTGITVTAFVLGFIAFLPLIIISSPIWIPLFLIVTGFLSLAGLILATGAVVSWLYRYFKGMHPLRSDQVDYARSRIHDTAAHVKDYAGGYFHGTLKDAAPGA

SEQ ID NO: 6 - protein, 191 aa, Arabidopsis thaliana
MANVDRDRRVHVDRTDKRVHQPNYEDDVGFGGYGGYGAGSDYKSRGPSTNQILALIAGVPIGGTLLTLAGLTLAGSVIGLLVSIPLFLLFSPVIVPAALTIGLAVTGILASGLFGLTGLSSVSWVLNYLRGTSDTVPEQLDYAKRRMADAVGYAGMKGKEMGQYVQDKAHEARETEFMTETHEPGKARRGS

SEQ ID NO: 7 - protein, 199 aa, Arabidopsis thaliana; identical to SEQ ID NO: 2

SEQ ID NO: 8 - protein, 183 aa, Arabidopsis thaliana; identical to SEQ ID NO: 4

SEQ ID NO: 9 - protein, 191 aa, Arabidopsis thaliana; identical to SEQ ID NO: 6

SEQ ID NO: 10 - protein, 183 aa, Arabidopsis thaliana; identical to SEQ ID NO: 4

SEQ ID NO: 11 - protein, 199 aa, Arabidopsis thaliana; identical to SEQ ID NO: 2

SEQ ID NO: 12 - protein, 191 aa, Arabidopsis thaliana
  misc_feature (160)..(160): Xaa can be any naturally occurring amino acid
MANVDRDRRVHVDRTDKRVHQPNYEDDVGFGGYGGYGAGSDYKSRGPSTNQILALIAGVPIGGTLLTLAGLTLAGSVIGLLVSIPLFLLFSPVIVPAALTIGLAVTGILASGLFGLTGLSSVSWVLNYLRGTSDTVPEQLDYAKRRMADAVSYAGMKGKXMGQYVQDKAHEARETEFMTETHEPGKARRGS

SEQ ID NO: 13 - protein, 141 aa, Arabidopsis thaliana
MADQTRTHHEMISRDSTQEAHPKARQMVKAATAVTAGGSLLVLSGLTLAGTVVALTVATPLLVIFSPVLVPAVVTVALIITGFLASGGFGIAAITAFSWLYRHMTGSGSDKIENARMKVGSRVQDTKYGQHNIGVQHQQVS

SEQ ID NO: 14 - protein, 167 aa, Arabidopsis thaliana
QKQHRTLNPYSLRKRKKEMADHQQHQQQQQPIMRSLHESSPSTRQIVRFVTAATIGLSLLVLSGLTLTGTVIGLIVATPLMVLFSPVLVPAVITIGLLTMGFLFSGGCGVAAATALTWIYKYVTGKHPMGADKVDYARMRIAEKAKELGHYTHSQPQQTHQTTTTTH

SEQ ID NO: 15 - protein, 166 aa, Arabidopsis thaliana
MAERFSSGEAQYWPNYGSTATTTVSNSPISSFFHQLRSHSPTSSQLFGFLALFISTGILLFLLGVSVTAAVLGFIVFLPLIIISSPIWIPVFVVVGGFLTVSGFLVGTVALVSWTYRYFRGMHPVGSNQMDYARSRIYDTASHVKDYAREYGGYFHGRAKDAAPGA

SEQ ID NO: 16 - protein, 166 aa, Arabidopsis thaliana; identical to SEQ ID NO: 15

SEQ ID NO: 17 - protein, 169 aa, Arabidopsis thaliana; identical to SEQ ID NO: 5

SEQ ID NO: 18 - protein, 173 aa, Arabidopsis thaliana
MADTARGTHHDIIGRDQYPMMGRDRDQYQMSGRGSDYSKSRQIAKAATAVTAGGSLLVLSSLTLVGTVIALTVATPLLVIFSPILVPALITVALLITGFLSSGGFGIAAITVFSWIYKYATGEHPQGSDKLDSARMKLGSKAQDLKDRAQYYGQQHTGGEHDRDRTRGGQHTT

SEQ ID NO: 19 - protein, 226 aa, Arabidopsis lyrata subsp. Lyrata
MFSFLILLLEVYKVVIAVVASIVFFVFSGLTLAGSAVALTVTTPLFIIFSPILVPATIATALLTTGFTAGGALGATAIALIRRSMGVKSKNNIPATGAPPTMFAQTPFNLTPKINYEGTFKGSWGGTSSPQAAPNFSYGGTWTATWGGRSFTGKFGDQSGGGGSTPEAAGAAADAGAGAGAGAGAGAGAGAGAAGAGAGAGAGAGAPGGAGTGTPAPPGKAGSKKK

SEQ ID NO: 20 - protein, 141 aa, Arabidopsis thaliana
MADQTRTHHEMISRDSTQEAHPKARQMVKAATAVTAGGSLLVLSGLTLAGTVIALTVATPLLVIFSPVLVPAVVTVALIITGFLASGGFGIAAITAFSWLYRHMTGSGSDKIENARMKVGSRVQDTKYGQHNIGVQHQQVS

SEQ ID NO: 21 - protein, 191 aa, Arabidopsis thaliana; identical to SEQ ID NO: 6

SEQ ID NO: 22 - protein, 199 aa, Arabidopsis thaliana; identical to SEQ ID NO: 2

SEQ ID NO: 23 - protein, 169 aa, Arabidopsis thaliana; identical to SEQ ID NO: 5

SEQ ID NO: 24 - protein, 32 aa, Arabidopsis thaliana
QNKAQDVKQYDISKPHDTTTKGHETQGRTTAA

SEQ ID NO: 25 - protein, 173 aa, Arabidopsis thaliana; identical to SEQ ID NO: 18

SEQ ID NO: 26 - protein, 191 aa, Arabidopsis thaliana
MANVDRDRRVHVDRTDKRVHQPNYEDDVGFGGYGGYGAGSDYKSRGPSTNQILALIAGVPIGGTLLTLAGLTLAGSVIGLLVSIPLFLLFSPVIVPAALTIGLAVTGILASGLFGLTGLSSVSWVLNYLRGTSDTVPEQLDYAKRRMADAVGYAGMKGKEMGQYVQDKAHEARETEFMTETHEPGKARRGP

SEQ ID NO: 27 - protein, 199 aa, Arabidopsis thaliana
MADTHRVDRTDRHFQFQSPYEGGRGQGQYEGDRGYGGGGYKSMMPESGPSSTQVLSLLIGVPVVGSLLALAGLLLAGSVIGLMVALPLFLLFSPVIVPAGLTIGLAMTGFLASGMFGLTGLSSISWVMNYLRGTKRTVPEQLEYAKRRMADAVGYAGQKGKEMGQHVQNKAQDVKQYDISKPHDTTTKGHETQGGTTAA

SEQ ID NO: 28 - protein, 191 aa, Arabidopsis thaliana; identical to SEQ ID NO: 6

SEQ ID NO: 29 - protein, 141 aa, Arabidopsis thaliana; identical to SEQ ID NO: 20

SEQ ID NO: 30 - protein, 173 aa, Arabidopsis thaliana; identical to SEQ ID NO: 18

SEQ ID NO: 31 - protein, 173 aa, Arabidopsis thaliana; identical to SEQ ID NO: 18

SEQ ID NO: 32 - protein, 153 aa, Arabidopsis thaliana
MAPFPLSLIFGKKKRRRDDEIRRQKPTLKGVMTAFFATEAAICLLLLAGISLTGTAVALFASMPLFLVFSPVLVPAGIATTILASGLMAGGTSGVSGLTILMWLYKKYTGRDFPIKIPGAAAAGGAAPAAPAAPAPAAPAAKPAAKPAAKPGA

SEQ ID NO: 33 - protein, 173 aa, Arabidopsis thaliana; identical to SEQ ID NO: 18

SEQ ID NO: 34 - protein, 199 aa, Arabidopsis thaliana; identical to SEQ ID NO: 2

SEQ ID NO: 35 - protein, 191 aa, Arabidopsis thaliana; identical to SEQ ID NO: 6

SEQ ID NO: 36 - protein, 141 aa, Arabidopsis thaliana; identical to SEQ ID NO: 20

SEQ ID NO: 37 - protein, 173 aa, Arabidopsis thaliana; identical to SEQ ID NO: 18

SEQ ID NO: 38 - protein, 199 aa, Arabidopsis thaliana; identical to SEQ ID NO: 27

SEQ ID NO: 39 - protein, 191 aa, Arabidopsis thaliana; identical to SEQ ID NO: 6

SEQ ID NO: 40 - protein, 153 aa, Arabidopsis thaliana; identical to SEQ ID NO: 32

SEQ ID NO: 41 - protein, 175 aa, Brassica napus
RRDQYPRDRDQYSMIGRDRDKYSMIGRDRDQYNMYGRDYSKSRQIAKAVTAVTAGGSLLVLSSLTLVGTVIALTVATPLLVIFSPILVPALITVALLITGFLSSGGFGIAAITVFSWIYKYATGEHPQGSDKLDSARMKLGGKVQDMKDRAQYYGQQQTGGEDDRDRTRGTQHTT

SEQ ID NO: 42 - protein, 113 aa, Brassica napus
  misc_feature (1)..(1): Xaa can be any naturally occurring amino acid
XIHLQPQYEGDVGYGYGYGGRADXKSRGPSKNQIVALIVGVPVGGSLLALAGLTLAGSVIGLMLSVPLFLLFSPVIVPAAIXXGLAVTAILASGLFGXXXLSSVVWXLNYLRG

SEQ ID NO: 43 - protein, 216 aa, Brassica oleracea
RFFRMFSFIFPLLNVIKLIIASVTSLVCLAFSCVTLGGSAVALIVSTPLFIIFSPILVPATIATTLLASGLMAGTTLGLTGIGLITGLVRTAGGVTLAESPIRRIIINRIKARLGGGGGSRLAMLKKILGLIKKLRGMSSGGAAPALKQHQQLRPRMELHPRHLHRPNKERWFMLFQYVAHKNCVIINLRIYDSETKKKIALLLSFIQYSFLCNNV

SEQ ID NO: 44 - protein, 108 aa, Brassica oleracea
MFFQIIQGVFTGVEALALLAFAGITLGGSAVGLALSTPLFILFSPILVPATIATTLLTTGFTTSGGLGMVALRIFWKLFKRLRKKGKGTPKIPGLAPGAPDSDPVSGG

SEQ ID NO: 45 - protein, 353 aa, Brassica oleracea
MLSSLIQIFQVFQVTSAVVVTAVLFALAGITLAGSVVGLIVATPLFVIFSPVLVPATIASTLLATNLSAGALFGVTAAALIVWLLKHRMGVHPKNNPPPAGAPPTEAAKPTDEPAEGATDKPKDNPTGGAADKPGDKPAGGAADNSGGKSDGGETDKPESKPAGGPVNKPKDKPAGGPTDKPGSKPADKPAGGPTDKPESKPAGDASNKPKDKPTGEPTDKPESKPAREASNKRKDKPAGGPTDKRESKPAGEVSHKPKDKSAGGPTTKPESKPAGEVSHKPKDKSAGGPTDKPGNKPVGGPADKPKDNPAGRPTDKPTGGTENKPAGEAANKPIGKPKNKPAGENKPPAWYS

SEQ ID NO: 46 - protein, 361 aa, Brassica oleracea
MRNEIQNETAQTDQTQGSMFSFFDLFPFLLPMFEVIKMVVASVASVVYLGFAGVTLSGSAVALAVSTPLFIIFSPILLPAIAATTVLAAGLGSKKVAAAPAASPSLSLLGIPESIKPSNVIPESIKPSNIIPESIKPSNIIPESVKPSNIKDKIKDTIGKVKNKINAKKEEKSKGKSEDSSKGKGKSKGEDTTTDEDKPGSGGKHGKGESKHGKGESTHGKGGKHGSEGSSMDEGKHGGKHGSGGSPMGGGKHGSGGKHESGGSPMGGGKHGSGGKHESGGASMGGGKHGSGGKHGSEGKHGGEGSSMGKNSQSKNKKEFHYRGQAMDASSTSESSDGSSDGSSDGSSSDGSSHGSGGKHI

SEQ ID NO: 47 - protein, 186 aa, Brassica oleracea
MSEELVRHESHSQASIFSRFFRMFSFIFPLLNVIKLIIASVTSLVCLAFSCVTLGGSAVALIVSTPLFIIFSPILVPATIATTLLASGLMAGTTLGLTGIGLITGLVRTAGGVTLAESPIRRIIINRIKARLGGGGGSRLAMLKKILGLIKKLRGMSSGGAAPAAEAAPAAAPADGAAPAAAPAPT

SEQ ID NO: 48 - protein, 171 aa, Brassica oleracea
MFSFLSPLLDVIKVVVASVTSVVLFVFAGLTLSGSAVALVVSTPLFLIFSPILVPATIATTLLASGVTAGATLGITAISLIMGLIKTAEGSSLARLAQTPLKLFKFSGGFGGSWGGKPFSGTFGNKGSQSSGNIPGWLKNLLNGIPGGGAAPAAGGAAPAPAAPAPAAPPG

SEQ ID NO: 49 - protein, 178 aa, Brassica rapa
PRDRDQYSMIGRDRDKYSMIGRDRDQYNMYGRDYSKSRQIAKAVTAVTAGGSLLVLSSLTLVGTVIALTVATPLLVIFSPILVPALITVALLITGFLSSGGFGIAAITVFSWIYKYATGEHPQGSDKLDSARMKLGGKVQDMKDRAQYYGQQHTGGYGQQQTGGEHDRDRTRGTQHTT

SEQ ID NO: 50 - protein, 175 aa, Brassica napus; identical to SEQ ID NO: 41

SEQ ID NO: 51 - protein, 195 aa, Brassica napus
MTDTARTHHDITSRDQYPRDRDQYSMIGRDRDQYSMMGRDRDQYNMYGRDYSKSRQIAKAVTAVTAGGSLLVLSSLTLVGTVIALTVATPLLVIFSPILVPALITVAMLITGFLSSGGFGIAAITVFSWIYKYATGEHPQGSDKLDSARMKLGSKAQDLKDRAQYYGQQHTGGYGQQHTGGEHDRDRTRGTQHTT

SEQ ID NO: 52 - protein, 183 aa, Brassica napus
PARTHHDITTRDQYPLISRDRDQYGMIGRDQYNMSGQNYSKSRQIAKATTAVTAGDSLLVLSSLTLVGTVIALIVATPLLVIFSPILVPALITVALLITGFLSSGAFGIAAITVFSWIYKYATGEHPQGSDKLDSARMKLGSKAQDMKDRAYYYGQQHTGEEHDRDRDHRTDRDRTRGTQHTT

SEQ ID NO: 53 - protein, 157 aa, Brassica napus
MGILRKKKHERNASFKSVLTSILATQAATFLLLISGVSLAGTAAAFIATMPLFVVFSPILVPAGITTGLLTTGLAAAGGAGATAVTIILWLYKQATGKEPPAVLSKVLKKIIPGAAAAPRAAPAAAPAAAPAAAPAAAPAPKPAAAPAPKPAAPPAL

SEQ ID NO: 54 - protein, 165 aa, Brassica napus
MGILRKKKHERKPSFKSVLTAILATHAATFLLLIAGVSLAGTAAAFIATMPLFVVFSPILVPAGITTGLLTTGLAAAGGAGATAVTIILWLYKRATGKAPPKVLEKVLKKIIPGAAAAPAAAPGAAPAAAPAAAPAVAPAAAPAAAPAPKPAAPPAPKPAAAPSI

SEQ ID NO: 55 - protein, 207 aa, Brassica napus
MEEEIQNETAQTLSQREGRMFSFLFPVLEVIKVVMASVASVVFLGFGGVTLACSAVALAVSTPLFIIFSPILVPATIATTLLATGLGAGTTLGVTGMGLLMRLIKHPGKEGAASAPAAQPSFLSLLEMPNFIKSKMLERLIHIPGVGKKSEGRGESKGKKGKSEHGRGKHEGEGKSKGRKGKSRGKDKDKKKGKGSRKGSSDDDESS

SEQ ID NO: 56 - protein, 84 aa, Brassica napus
FIFSPILVPATIATALLATGFTASGSIGAMAITIFMWLFKKITGKNPPKIPFLTPKIQLRKGEKRRIKKRKKKKRMSRPFRTMG

SEQ ID NO: 57 - protein, 92 aa, Brassica napus
FSPILVPAGVATGLLATGLAAAGSSGAMAVSLILWVIKRVTGKEPPKIMSKVLRKVFPGGGTPSKLAAKPEAEPARAPAAAPSAAPAAAPST

SEQ ID NO: 58 - protein, 63 aa, Brassica napus
FSPILVPAGITTGLLAMGLATSGGSGLTALSIMSWLKKLTVKELPKIRVKRRTGSPGGHGSTE

SEQ ID NO: 59 - protein, 91 aa, Brassica napus
IFSPILVPAGVATGLLATGLAAAGSSGAVAVSLILWVFKRVTGKEPPKIMSKVLKKVSPGGGTPSKLAAKPEAEPARTSAAPSATAAAPST

SEQ ID NO: 60 - protein, 208 aa, Brassica napus
SMEEEIQNETAQTLSQREGRMFSYLFPVLEVIKVVMASVASVVFLGFGGVTLACSAVALAVSTPLFIIFSPILVPATIATTLLATGLGAGTTLGVSGMGLLMRLIKHPGKEGAASAPAAQPSFLSLLEMPNFIKSKMLERLIHIPGVGKKSEGRGESKGEKGKSEHGRGKHEGDGKSKGRKGKSRGKDKDKKKGKGSRKGSSDDDESS

SEQ ID NO: 61 - protein, 65 aa, Brassica napus
FSPILVPATIATTLLTTGFTTSGGLGIVALRIFWKLFKRLRKRGKGTPKISRIGPGPDSNSVSGG

SEQ ID NO: 62 - protein, 375 aa, Brassica napus
MKEEIQNETAQTQLQREGRMFSFLFPVIEVIKVVMASVASVVFLGFGGVTLACSAVALAVSTPLFIIFSPILVPATIATTLLATGLGAGTTLGVTGMGLLMRLIKHPGKEGAASAPAAQPSFLSLLEMPNFIKSKMLERLIHIPGVGKKSEGRGESKGKKGKKGKSEHGRGKHEGEGKSKGRKGHRMGVNPENNPPPAGAPPTGSPPAAPAAPEAPAAPAAPAAPAAPAAPAAPAAPEDPAAPAAPEAPATPAAPPAPAAAPAPAAPAAPPAPAAPPRPPSFLSLLEMPSFIKSKLIEALINIPGFGKKSNDRGKSKGGKKSKGKGKSNGRGKHEGEGKSKSRKSKSRGKDKEKSKGKGIFGRSSRKGSSDDESS

SEQ ID NO: 63 - protein, 183 aa, Brassica napus; identical to SEQ ID NO: 52

SEQ ID NO: 64 - protein, 195 aa, Brassica napus; identical to SEQ ID NO: 51

SEQ ID NO: 65 - protein, 175 aa, Brassica napus; identical to SEQ ID NO: 41

SEQ ID NO: 66 - protein, 183 aa, Brassica napus
QASIFSRFFRMFSFIFPFVNVIKLIIASVTSLVCLAFSCVALGGSAVALIVSTPLFIMFSPILVPATIATTLLASGLMAGTTLGLTGIGLIMGLVRTAGGVSLLQSPLRKIIVNRIKARLGGGGGGSRLARLKKILGLLNKLRGMGAGGAAAPAAEPAPAAEAAPAAEAAPAAAPAAAPAAAP

SEQ ID NO: 67 - protein, 113 aa, Brassica napus
  misc_feature (1)..(1): Xaa can be any naturally occurring amino acid
XIHLQPQYEGDVGYGYGYGGRADYKSRGPSXNQIVALIVGVPVGGSLLALAGLTLAGSVIGLMLSVPLFLLFSPVIVPAAIXXGLAVTAILASGLFGXXXLSSVVWXLNYLRG

SEQ ID NO: 68 - protein, 195 aa, Brassica napus; identical to SEQ ID NO: 51

SEQ ID NO: 69 - protein, 183 aa, Brassica napus; identical to SEQ ID NO: 52

SEQ ID NO: 70 - protein, 165 aa, Brassica napus; identical to SEQ ID NO: 54

SEQ ID NO: 71 - protein, 175 aa, Brassica napus; identical to SEQ ID NO: 41

SEQ ID NO: 72 - protein, 375 aa, Brassica napus; identical to SEQ ID NO: 62

SEQ ID NO: 73 - protein, 187 aa, Brassica napus
MTDTARTHHDITSRDQYPRDRDQYSMIGRDRDKYSMIGRDRDQYNMYGRDYSKSRQIAKAVTAVTAGGSLLVLSSLTLVGTVIALTVATPLLVIFSPILVPALITVALLITGFLSSGGFGIAAITVFSWIYKYATGEHPQGSDKLDSARMKLGGKVQDMKDRAQYYGQQQTGGEHDRDRTRGTQHTT

SEQ ID NO: 74 - protein, 195 aa, Brassica napus; identical to SEQ ID NO: 51

SEQ ID NO: 75 - protein, 168 aa, Daucus carota
  misc_feature (55)..(55): Xaa can be any naturally occurring amino acid
NKFTLSNLISVDFMAMYQSPQFTRHHDALQPAALNSSGQGHHSSHHRRSVMLLSXLTLVATVIGLVIATPVMVIFSPVLVPAGLPAAPARRVSHGGGAGGHRAFVLFWMYRYTAGKHPIGANQLDFAATRLRMRKEKGXIWGMSRFRLFRGVEEVVRRXGNDEGFLAV

SEQ ID NO: 76 - protein, 168 aa, Daucus carota; identical to SEQ ID NO: 75
  misc_feature (55)..(55): Xaa can be any naturally occurring amino acid

SEQ ID NO: 77 - protein, 187 aa, Zea mays
MADRDRSGIYGGAHATYGQQQQQGGGGRPMGEQVKKGMLHDKGPTASQALTVATLFPLGGLLLVLSGLALTASVVGLAVATPVFLIFSPVLVPAALLIGTAVMGFLTSGALGLGGLSSLTCLANTARQAFQRTPDYVEEARRRMAEAAAQAGHKTAQAGQAIQGRAQEAGTGGGAGAGAGGGGRASS

SEQ ID NO: 78 - protein, 187 aa, Zea mays; identical to SEQ ID NO: 77

SEQ ID NO: 79 - protein, 156 aa, Zea mays
MADHHRGATGGGGGYGDLQRGGGMHGEAQQQQKQGAMMTALKAATAATFGGSMLVLSGLILAGTVIALTVATPVLVIFSPVLVPAAIALALMAAGFVTSGGLGVAALSVFSWMYKYLTGKHPPAADQLDHAKARLASKARDVKDAAQHRIDQAQGS

SEQ ID NO: 80 - protein, 175 aa, Zea mays
MADRDRSGIYGGGAYGQQQGRPPMGEQVKGMIHDKGPTASQALTVATLFPLGGLLLVLSGLALAASTVGLAVATPVFLLFSPVLVPAALLIGTAVAGFLTSGALGLGGLSSLTCLANTARQAFQRTPDYVEEARRRMAEAAAHAGHKTAQAGHGIQSKAQEAGAGTGAGGGRTSS

SEQ ID NO: 81 - protein, 156 aa, Zea mays; identical to SEQ ID NO: 79

SEQ ID NO: 82 - protein, 187 aa, Zea mays; identical to SEQ ID NO: 77

SEQ ID NO: 83 - protein, 156 aa, Zea mays; identical to SEQ ID NO: 79

SEQ ID NO: 84 - protein, 175 aa, Zea mays; identical to SEQ ID NO: 80

SEQ ID NO: 85 - DNA, 516 bp, Artificial Sequence
  antisense sequence without the primer (i.e. just cDNA)
agtgtgttgaccaccacgagtacggtcacggtcatgttccccaccagtatgttgctgtccgtagtactgagctctgtctttcagatcctgagctttgcttcccaacttcatccttgcactgtccaacttgtctgatccctgtgggtgctctcccgttgcgtacttgtaaatccaagagaaaacggttatagcggcaatgccaaaccctccagaggaaagaaaaccggtgatgaggagtgcaactgtgatgagagccgggacaaggattgggctgaagataacgagcagaggtgttgcaacagtcaaagctatgacagttccaacaagggtaaggctggagagaacaaggagggaaccaccagctgtgacagcagttgcagctttagcaatctgcctagacttggagtagtcagatcctcgtccggacatctggtactggtctcggtctcggcccatcatcgggtactggtctctgccgatgatatcgtgatgggttcctctagcagtatcggccat

SEQ ID NO: 86 - DNA, 540 bp, Artificial Sequence
  antisense sequence with primer sequence
agccatactagtagtgtgttgaccaccacgagtacggtcacggtcatgttccccaccagtatgttgctgtccgtagtactgagctctgtctttcagatcctgagctttgcttcccaacttcatccttgcactgtccaacttgtctgatccctgtgggtgctctcccgttgcgtacttgtaaatccaagagaaaacggttatagcggcaatgccaaaccctccagaggaaagaaaaccggtgatgaggagtgcaactgtgatgagagccgggacaaggattgggctgaagataacgagcagaggtgttgcaacagtcaaagctatgacagttccaacaagggtaaggctggagagaacaaggagggaaccaccagctgtgacagcagttgcagctttagcaatctgcctagacttggagtagtcagatcctcgtccggacatctggtactggtctcggtctcggcccatcatcgggtactggtctctgccgatgatatcgtgatgggttcctctagcagtatcggccatggaagcttaata

SEQ ID NO: 87 - DNA, 516 bp, Artificial Sequence
  sense sequence without primer (i.e. just cDNA)
atggccgatactgctagaggaacccatcacgatatcatcggcagagaccagtacccgatgatgggccgagaccgagaccagtaccagatgtccggacgaggatctgactactccaagtctaggcagattgctaaagctgcaactgctgtcacagctggtggttccctccttgttctctccagccttacccttgttggaactgtcatagctttgactgttgcaacacctctgctcgttatcttcagcccaatccttgtcccggctctcatcacagttgcactcctcatcaccggttttctttcctctggagggtttggcattgccgctataaccgttttctcttggatttacaagtacgcaacgggagagcacccacagggatcagacaagttggacagtgcaaggatgaagttgggaagcaaagctcaggatctgaaagacagagctcagtactacggacagcaacatactggtggggaacatgaccgtgaccgtactcgtggtggtcaacacact

SEQ ID NO: 88 - DNA, 540 bp, Artificial Sequence
  sense sequence with primer
tattaagcttccatggccgatactgctagaggaacccatcacgatatcatcggcagagaccagtacccgatgatgggccgagaccgagaccagtaccagatgtccggacgaggatctgactactccaagtctaggcagattgctaaagctgcaactgctgtcacagctggtggttccctccttgttctctccagccttacccttgttggaactgtcatagctttgactgttgcaacacctctgctcgttatcttcagcccaatccttgtcccggctctcatcacagttgcactcctcatcaccggttttctttcctctggagggtttggcattgccgctataaccgttttctcttggatttacaagtacgcaacgggagagcacccacagggatcagacaagttggacagtgcaaggatgaagttgggaagcaaagctcaggatctgaaagacagagctcagtactacggacagcaacatactggtggggaacatgaccgtgaccgtactcgtggtggtcaacacactactagtatggct

SEQ ID NO: 89 - DNA, 276 bp, Artificial Sequence
  intron sequence with SpeI site and primer
actagtgatttacaagtaagcacacatttatcatcttacttcataattttgtgcaatatgtgcatgcatgtgttgagccagtagctttggatcaatttttttggtcgaataacaaatgtaacaataagaaattgcaaattctagggaacatttggttaactaaatacgaaatttgacctagctagcttgaatgtgtctgtgtatatcatctatataggtaaaatgcttggtatgatacctattgattgtgaataggtacgcaacgggagaactagt

SEQ ID NO: 90 - DNA, 264 bp, Artificial Sequence
  loop sequence plus primer (no SpeI site)
gatttacaagtaagcacacatttatcatcttacttcataattttgtgcaatatgtgcatgcatgtgttgagccagtagctttggatcaatttttttggtcgaataacaaatgtaacaataagaaattgcaaattctagggaacatttggttaactaaatacgaaatttgacctagctagcttgaatgtgtctgtgtatatcatctatataggtaaaatgcttggtatgatacctattgattgtgaataggtacgcaacgggaga

SEQ ID NO: 91 - DNA, 240 bp, Artificial Sequence
  loop sequence without primer
gtaagcacacatttatcatcttacttcataattttgtgcaatatgtgcatgcatgtgttgagccagtagctttggatcaatttttttggtcgaataacaaatgtaacaataagaaattgcaaattctagggaacatttggttaactaaatacgaaatttgacctagctagcttgaatgtgtctgtgtatatcatctatataggtaaaatgcttggtatgatacctattgattgtgaatag

SEQ ID NO: 92 - DNA, 32 bp, Artificial Sequence
  forward primer NTD
tattaagcttccatggccgatactgctagagg

SEQ ID NO: 93 - DNA, 32 bp, Artificial Sequence
  reverse primer CTR
agccatactagtagtgtgttgaccaccacgag

SEQ ID NO: 94 - DNA, 516 bp, Artificial Sequence; identical to SEQ ID NO: 87
  Atol 1 cDNA

SEQ ID NO: 95 - DNA, 36 bp, Artificial Sequence
  forward primer IntronD
ttttactagtgatttacaattaagcacacatttatc

SEQ ID NO: 96 - DNA, 33 bp, Artificial Sequence
  reverse primer IntronR
ctgtactagttctcccgttgcgtacctattcac

SEQ ID NO: 97 - DNA, 540 bp, Artificial Sequence; identical to SEQ ID NO: 86
  antisense construct

SEQ ID NO: 98 - DNA, 1080 bp, Artificial Sequence
  hairpin construct
tattaagcttccatggccgatactgctagaggaacccatcacgatatcatcggcagagaccagtacccgatgatgggccgagaccgagaccagtaccagatgtccggacgaggatctgactactccaagtctaggcagattgctaaagctgcaactgctgtcacagctggtggttccctccttgttctctccagccttacccttgttggaactgtcatagctttgactgttgcaacacctctgctcgttatcttcagcccaatccttgtcccggctctcatcacagttgcactcctcatcaccggttttctttcctctggagggtttggcattgccgctataaccgttttctcttggatttacaagtacgcaacgggagagcacccacagggatcagacaagttggacagtgcaaggatgaagttgggaagcaaagctcaggatctgaaagacagagctcagtactacggacagcaacatactggtggggaacatgaccgtgaccgtactcgtggtggtcaacacactactagtatggctagccatactagtagtgtgttgaccaccacgagtacggtcacggtcatgttccccaccagtatgttgctgtccgtagtactgagctctgtctttcagatcctgagctttgcttcccaacttcatccttgcactgtccaacttgtctgatccctgtgggtgctctcccgttgcgtacttgtaaatccaagagaaaacggttatagcggcaatgccaaaccctccagaggaaagaaaaccggtgatgaggagtgcaactgtgatgagagccgggacaaggattgggctgaagataacgagcagaggtgttgcaacagtcaaagctatgacagttccaacaagggtaaggctggagagaacaaggagggaaccaccagctgtgacagcagttgcagctttagcaatctgcctagacttggagtagtcagatcctcgtccggacatctggtactggtctcggtctcggcccatcatcgggtactggtctctgccgatgatatcgtgatgggttcctctagcagtatcggccatggaagcttaata

SEQ ID NO: 99 - DNA, 1332 bp, Artificial Sequence
  hairpin + intron construct
tattaagcttccatggccgatactgctagaggaacccatcacgatatcatcggcagagaccagtacccgatgatgggccgagaccgagaccagtaccagatgtccggacgaggatctgactactccaagtctaggcagattgctaaagctgcaactgctgtcacagctggtggttccctccttgttctctccagccttacccttgttggaactgtcatagctttgactgttgcaacacctctgctcgttatcttcagcccaatccttgtcccggctctcatcacagttgcactcctcatcaccggttttctttcctctggagggtttggcattgccgctataaccgttttctcttggatttacaagtacgcaacgggagagcacccacagggatcagacaagttggacagtgcaaggatgaagttgggaagcaaagctcaggatctgaaagacagagctcagtactacggacagcaacatactggtggggaacatgaccgtgaccgtactcgtggtggtcaacacactactagtgatttacaagtaagcacacatttatcatcttacttcataattttgtgcaatatgtgcatgcatgtgttgagccagtagctttggatcaatttttttggtcgaataacaaatgtaacaataagaaattgcaaattctagggaacatttggttaactaaatacgaaatttgacctagctagcttgaatgtgtctgtgtatatcatctatataggtaaaatgcttggtatgatacctattgattgtgaataggtacgcaacgggagaactagtagtgtgttgaccaccacgagtacggtcacggtcatgttccccaccagtatgttgctgtccgtagtactgagctctgtctttcagatcctgagctttgcttcccaacttcatccttgcactgtccaacttgtctgatccctgtgggtgctctcccgttgcgtacttgtaaatccaagagaaaacggttatagcggcaatgccaaaccctccagaggaaagaaaaccggtgatgaggagtgcaactgtgatgagagccgggacaaggattgggctgaagataacgagcagaggtgttgcaacagtcaaagctatgacagttccaacaagggtaaggctggagagaacaaggagggaaccaccagctgtgacagcagttgcagctttagcaatctgcctagacttggagtagtcagatcctcgtccggacatctggtactggtctcggtctcggcccatcatcgggtactggtctctgccgatgatatcgtgatgggttcctctagcagtatcggccatggaagcttaata

(19) United States
(12) Patent Application Publication
(10) Pub. No.: US 2008/0104725 A1
(43) Pub. Date: May 1, 2008

(54) Methods For The Modulation of Oleosin Expression In Plants

(76) Inventors: Rodrigo M. Siloto, Calgary (CA); Maurice M. Moloney, Calgary (CA)

Correspondence Address: BERESKIN AND PARR, 40 KING STREET WEST, BOX 401, TORONTO, ON, M5H 3Y2, US

(21) Appl. No.: 11/576,389

(22) PCT Filed: Oct. 6, 2005
(86) PCT No.: PCT/CA05/01529

§ 371 Date: Nov. 21, 2007

Related U.S. Application Data
(60) Provisional application No. 60/615,939, filed on Oct. 6, 2004.

(51) Int. Cl. C12N 15/82; A01H 5/00; C11B 1/00; C12N 15/00; C12N 15/11

(52) U.S. Cl. 800/278; 435/320.1; 536/23.6; 554/8; 554/9; 800/298; 800/310; 800/312; 800/320.1

(57) ABSTRACT

Methods to modulate oleosin expression levels in plants are provided. Specifically, methods for preparing seed derived products from seed, in which the composition of seed storage reserves, notably the seed lipid and protein contents, have been altered. In particular the present invention provides methods for preparing seed derived products from seed, in which the seed reserves have been altered by modulation of oleosin gene expression and more particularly the suppression of oleosin gene expression.

### FIELD OF THE INVENTION

The present invention relates to plant genetic engineering methods. More specifically, the present invention relates to methods to modulate the expression levels of oleosin proteins in plants.

### BACKGROUND OF THE INVENTION

Plant seeds represent an important source of nutrients for both human and animal use. For example, plant seed proteins represent a major component of animal feed and plant seed oil is used for the production of vegetable oil which is used extensively for human consumption.

In seeds, the water insoluble oil fraction is stored in discrete subcellular structures variously known in the art as oil bodies, oleosomes, lipid bodies or spherosomes (Huang, 1992 Ann. Rev. Plant Mol. Biol. 43: 177-200), having a diameter ranging between 0.5 and 2.0 micrometers (Tzen, 1993 Plant Physiol. 101: 267-276). Besides a mixture of oils (triacylglycerides), which chemically are defined as glycerol esters of fatty acids, oil bodies comprise phospholipids and a number of associated proteins, collectively termed oil body proteins. From a structural point of view, oil bodies are considered to be a triacylglyceride matrix encapsulated by a monolayer of phospholipids in which oil body proteins are embedded (Huang, 1992 Ann. Rev. Plant Mol. Biol. 43: 177-200). The seed oil present in the oil body fraction of plant species is a mixture of various triacylglycerides, of which the exact composition depends on the plant species from which the oil is derived.

Methodologies for the modulation of the lipid and protein constituents of plant seeds and consequently the nutritional value of plant seeds are well known. Such methodologies include traditional plant breeding, as well as genetic engineering based methodologies. However despite the availability of such methodologies, methods which demonstrably result in the modulation of the levels of oil body proteins in seed, especially oleosin proteins, which may constitute up to for example 8-20% in Brassica (Huang (1992) Annu Rev Plant Physiol Plant Mol Biol 43: 177-200 and Murphy and Cummins (1989) J. Plant Physiol 135: 63-69.) of the total seed protein, are limited.

The prior art provides Arabidopsis thaliana plant lines which were generated using an Agrobacterium T-DNA insertion mutant based methodology. Using this methodology over 225,000 independent genomic insertion events were created (Alsonso et al. (2003) Science 301: 653-657). To date within this population of plant lines, two Arabidopsis oleosin mutants have been identified. SM_{—}3-29875 contains a DNA insertion in the second exon of Atol1 (Tissier A. F. et al., Plant Cell 11: 1841-1852) and SALK_{—}072403 contains a single insertion in Atol2 (Alonso J. M. et al., Science 301: 653-657). While these Arabidopsis mutants display an ablation of the expression of the oleosin genes, the methodology used to generate these plant lines is random and does not allow for the specific suppression of a specific gene, nor does it allow for the generation of plant lines with a varying range of expression levels of a particular oleosin gene. T-DNA Agrobacterium insertion mutagenesis methodology also becomes increasingly unpractical when crop plants with a larger genome size are used.

Chaudhary S. (2002, Ph.D. Thesis. University of Calgary. Molecular biology of flax (Linum usitatissimum L) seed oleosin genes.) speculates that an anti-sense gene knock-out strategy may be employed to suppress the levels of endogenously present oleosin proteins, however no details are documented on how using this methodology such oleosin gene suppression might be achieved, or indeed whether oleosin suppression may be achieved.

Thus in view of the shortcomings of the prior art it is presently unclear how oleosin gene expression may be suppressed in plants other than by using a T-DNA Agrobacterium insertion methodology. Furthermore, it is unclear whether and how suppression of oleosin gene expression may be used to modulate the seed lipid and protein constituents in a plant seed. There is a need in the art to improve methods for the suppression of oleosins in plants.

### SUMMARY OF THE INVENTION

The present invention relates to methods for preparing seed derived products from seed, in which the composition of seed storage reserves, notably the seed lipid and protein contents, have been altered. In particular the present invention provides methods for preparing seed derived products from seed, in which the seed reserves have been altered by modulation of oleosin gene expression and more particularly the suppression of oleosin gene expression.

Accordingly, the present invention provides a method for preparing a plant seed derived product from plants seeds comprising:
(a) providing a chimeric nucleic acid construct comprising in the 5′ to 3′ direction of transcription as operably linked components:
   (i) a nucleic acid sequence capable of controlling expression in plant cells; and
   (ii) a nucleic acid sequence which upon transcription generates a RNA nucleic acid sequence that is complementary to a nucleic acid sequence encoding an oleosin mRNA or a fragment thereof;
(b) introducing the chimeric nucleic acid construct into a plant cell to obtain a transformed plant cell;
(c) regenerating from said transformed plant cell a transformed plant capable of setting seed;
(d) harvesting said seed wherein said seed has a modified oleosin profile; and
(e) preparing a plant seed derived product from said seed.

The present invention also provides a method to increase the protein content in plants seeds, comprising:
(a) providing a chimeric nucleic acid construct comprising in the 5′ to 3′ direction of transcription as operably linked components:
   (i) a nucleic acid sequence capable of controlling expression in plant cells; and
   (ii) a nucleic acid sequence which upon transcription generates a RNA nucleic acid sequence that is complementary to a nucleic acid sequence encoding an oleosin mRNA or a fragment thereof;
(b) introducing the chimeric nucleic acid construct into a plant cell to obtain a transformed plant cell; and
(c) regenerating from said transformed plant cell a transformed plant capable of setting seed wherein the protein content in said seed is increased as compared to a non-transformed plant.

The present invention also provides a method to decrease the lipid content in plants seeds, comprising:
(a) providing a chimeric nucleic acid construct comprising in the 5′ to 3′ direction of transcription as operably linked components:
   (i) a nucleic acid sequence capable of controlling expression in plant cells; and
   (ii) a nucleic acid sequence which upon transcription generates a RNA nucleic acid sequence that is complementary to a nucleic acid sequence encoding an oleosin mRNA or a fragment thereof;
(b) introducing the chimeric nucleic acid construct into a plant cell to obtain a transformed plant cell; and
(c) regenerating from said transformed plant cell a transformed plant capable of setting seed wherein the lipid content in said seed is decreased as compared to a non-transformed plant.

The seed obtained from these plants prepared in accordance with the present invention may be used as a source for the preparation of a variety of plant seed derived products.

Furthermore the present invention provides a method to suppress expression of an oleosin protein in a plant comprising:
(a) providing a chimeric nucleic acid construct comprising in the 5′ to 3′ direction of transcription as operably linked components:
   (i) a nucleic acid sequence capable of controlling expression in plant cells; and
   (ii) a nucleic acid sequence which upon transcription generates a RNA nucleic acid sequence that is complementary to a nucleic acid sequence encoding an oleosin mRNA or a fragment thereof;
(b) introducing the chimeric nucleic acid construct into a plant cell to obtain a transformed plant cell; and
(c) regenerating from said transformed plant cell a transformed plant, wherein expression of an oleosin protein is suppressed in said transformed plant.

In a preferred embodiment of the present invention, the nucleic acid sequence which upon transcription generates a RNA nucleic acid sequence that is complementary to a nucleic acid sequence encoding an oleosin mRNA is linked to a nucleic acid sequence encoding an oleosin. Accordingly, the present invention provides a method to suppress expression of an oleosin protein in a plant comprising:
(a) providing a chimeric nucleic acid construct comprising in the 5′ to 3′ direction of transcription as operably linked components:
   (i) a nucleic acid sequence capable of controlling expression in plant cells;
   (ii) a nucleic acid sequence which upon transcription generates a RNA nucleic acid sequence that is complementary to a nucleic acid sequence encoding an oleosin mRNA or a fragment thereof; and
   (iii) a nucleic acid sequence encoding an oleosin or a fragment thereof, wherein said nucleic acid sequence is complementary to the nucleic acid sequence provided in (ii);
(b) introducing the chimeric nucleic acid construct into a plant cell to obtain a transformed plant cell; and
(c) regenerating from said transformed plant cell a transformed plant wherein expression of an oleosin protein is suppressed in said transformed plant.

In a preferred embodiment of the present invention, the nucleic acid sequence capable of controlling expression in plant cells permits expression in plant seed cells and the transformed plant is a plant capable of setting seed. In a further preferred embodiment, the promoter is a seed-preferred promoter.

Accordingly the present invention provides a method to suppress expression of an oleosin protein in the seed of a plant comprising:
(a) providing a chimeric nucleic acid construct comprising in the 5′ to 3′ direction of transcription as operably linked components:
   (i) a nucleic acid sequence capable of controlling expression in plant seed cells; and
   (ii) a nucleic acid sequence which upon transcription generates a RNA nucleic acid sequence that is complementary to a nucleic acid sequence encoding an oleosin mRNA or a fragment thereof;
(b) introducing the chimeric nucleic acid construct into a plant cell to obtain a transformed plant cell;
(c) regenerating from said transformed plant cell a transformed plant; and
(d) growing the transformed plant into a mature plant capable of setting seed, wherein in the seed oleosin expression levels are suppressed.

In a preferred embodiment, the present invention provides a method to suppress expression of an oleosin protein in the seed of a plant comprising:
(a) providing a chimeric nucleic acid construct comprising in the 5′ to 3′ direction of transcription as operably linked components:
   (i) a nucleic acid sequence capable of controlling expression in plant seed cells;
   (ii) a nucleic acid sequence which upon transcription generates a RNA nucleic acid sequence that is complementary to a nucleic acid sequence encoding an oleosin mRNA or a fragment thereof; and
   (iii) a nucleic acid sequence encoding an oleosin or a fragment thereof, wherein said nucleic acid sequence is complementary to the nucleic acid sequence provided in (ii);
(b) introducing the chimeric nucleic acid construct into a plant cell to obtain a transformed plant cell;
(c) regenerating from said transformed plant cell a transformed plant; and
(d) growing the transformed plant into a mature plant capable of setting seed, wherein in the seed oleosin expression levels are suppressed.

In yet another preferred embodiment, the present invention provides a method for preparing a plant seed derived product from plant seeds comprising:
(a) providing a chimeric nucleic acid construct comprising in the 5′ to 3′ direction of transcription as operably linked components:
   (i) a nucleic acid sequence capable of controlling expression in plant seed cells;
   (ii) a nucleic acid sequence which upon transcription generates a RNA nucleic acid sequence that is complementary to a nucleic acid sequence encoding an oleosin mRNA sequence or fragment thereof; and
   (iii) a nucleic acid sequence encoding an oleosin or a fragment thereof, wherein said nucleic acid sequence is complementary to the nucleic acid sequence provided in (ii);
(b) introducing the chimeric nucleic acid construct into a plant cell;
(c) regenerating from said transformed plant cell a transformed plant capable of setting seed;
(d) harvesting said seed wherein said seed has a modified oleosin profile; and
(e) preparing a plant seed derived product from said seed.

In a further preferred embodiment the chimeric nucleic acid construct is introduced into the plant cell under nuclear genomic integration conditions. Under such conditions the chimeric nucleic acid sequence is stably integrated in the plant's genome.

Other features and advantages of the present invention will become readily apparent from the following detailed description. It should be understood, however, that the detailed description and the specific examples while indicating preferred embodiments of the invention, are given by way of illustration only, since various changes and modifications within the spirit and scope of the invention will become readily apparent to those skilled in the art from this detailed description.

### BRIEF DESCRIPTION OF THE DRAWINGS

The invention will now be described in relation to the drawings in which:

FIG. 1: Structure of RNA molecules that trigger post-transcriptional gene silencing. (a) Scheme of a complementary RNA strand to the target mRNA. (b) Scheme of a hairpin RNA structure. The molecule contain a self complementary region composed by a portion identical to the target mRNA (sense portion) and another portion complementary identical to the target mRNA (antisense portion). The hairpin RNA might contain the sense portion in the 3′ end and antisense portion in the 5′ end (left panel) or the antisense portion in the 3′ end and the sense portion in the 5′ end (right panel). (c) Scheme of a hairpin-loop RNA structure. The molecule contain a self complementary region composed by a portion identical to the target mRNA (sense portion), a region that is not identical or complementary to the target mRNA (loop) and another portion complementary to the target mRNA (antisense portion). The hairpin-loop RNA might contain the sense portion in the 3′ end followed by the loop portion, followed by the antisense portion in the 5′ end (left panel) or the antisense portion in the 3′ end, followed by the loop portion, followed by the sense portion in the 5′ end (right panel).

FIG. 2: Different configuration of cassettes used to suppress one or multiple oleosin genes. (a) Antisense cassette to suppress a single oleosin gene: a promoter fragment is fused to an oleosin coding region in inverted orientation followed by a terminator fragment. (b) Antisense cassette to suppress two oleosin genes: a promoter fragment is fused to two oleosin coding regions, both in inverted orientation, followed by a terminator fragment. (c) Hairpin cassette to suppress a single oleosin gene: a promoter fragment is fused to an oleosin coding region, followed by the same coding region in inverted orientation followed by a terminator fragment. (d) Another configuration of hairpin cassette: a promoter fragment is fused to an oleosin coding region in inverted orientation, followed by the same coding region in the upright orientation followed by a terminator fragment. (e) Hairpin-loop cassette to suppress a single oleosin gene: a promoter fragment is fused to an oleosin coding region, followed by a DNA fragment that is not related to the coding region (for example, an intron), followed by the same coding region in inverted orientation followed by a terminator fragment. (f) Hairpin-loop cassette to suppress a single oleosin gene: a promoter fragment is fused to an oleosin coding region inverted orientation, followed by an unrelated DNA fragment, followed by the same coding region in correct orientation, followed by a terminator fragment. (g) Hairpin-loop cassette to suppress three different oleosin genes: a promoter fragment is fused to three oleosin coding regions in tandem, followed by an unrelated DNA fragment, followed by the same three coding regions in the same order in inverse orientation, followed by a terminator fragment. (h) Hairpin-loop cassette to suppress three different oleosin genes: a promoter fragment is fused to three oleosin coding regions in tandem and reverse orientation, followed by an unrelated DNA fragment, followed by the same three coding regions in the same order in correct orientation, followed by a terminator fragment. (i) Hairpin-loop cassette to suppress three different oleosin genes: a promoter fragment is fused to the first oleosin coding regions in correct orientation, followed by the second oleosin coding region in inverse orientation, followed by the third oleosin coding region in correct orientation followed by an unrelated DNA fragment, followed by third coding region in inverse orientation, followed by the second coding region in correct orientation, followed by the first coding region I inverse orientation, followed by a terminator fragment.

FIG. 3: Scheme for construction of antisense and hairpin cassettes. The cDNA encoding for the 18 kDa oleosins from Arabidopsis thaliana (Atol1) (a) was obtained by PCR reaction with the primers NTD and CTR and inserted in the plasmid pSBS2090 (b) previously digested with the enzyme SwaI between the phaseolin promoter and terminator. The insertion provided the plasmid pAntisense (c) and pHairpin (d) that were selected according to the profile obtained with NcoI and HindIII/SalI.

FIG. 4: Scheme for construction of hairpin-loop cassette. The plasmid pHairpin (b) was digested with the enzymes KpnI and BamHI. The hairpin cassette was sub cloned in the vector pUC19 (a), generating the plasmid pUC-Hairpin (c). A fragment corresponding for the intron of Atol1 gene was amplified using the primers IntronD and Intron R (d). These primers created a restriction site for SpeI in each end. The fragment was purified, digested with SpeI and inserted in the plasmid pUC-Hairpin. The resulting plasmid is called pHairpin-intron (e).

FIG. 5: Scheme for construction of binary vectors carrying suppression cassettes. The plasmids pAntisense, pHairpin and pHairpin+intron (a) were digested with BamHI and KpnI. The cassettes were inserted in the binary vector pSBS3000 (b). The resulting plasmids were respectively called pSBS3000 Antisense, pSBS3000 Hairpin and pSBS3000 Hairpin+intron (c).

FIG. 6: Suppression of Atol1 oleosins in seeds from transgenic Arabidopsis lines. Oil bodies are extracted from seeds of Arabidopsis lines containing the suppression cassettes (lane 3) antisense, (lane 2) harpin (lane 1) harpin+intron. Oil body associated proteins are loaded in a SDS-PAGE 15% and stained with Comassie blue R250.

FIG. 7: In vivo comparison of oil bodies size in Arabidopsis lines. Panel “a” shows the oil bodies from wildtype (untransformed) Arabidopsis seeds. Panel “b” shows the oil bodies from Arabidopsis seeds containing the antisense suppression cassette. Panel “c” shows the oil bodies from Arabidopsis seeds containing the hairpin suppression cassette. Panel “d” shows the oil bodies from Arabidopsis seeds containing the hairpin+intron suppression cassette. The red circles represent oil bodies. The white bars indicate reference distances in “μm”.

FIG. 8: In vitro comparison of oil bodies size in Arabidopsis lines. Panel “A” shows the oil bodies from wildtype (untransformed) Arabidopsis seeds. Panel “B” shows the oil bodies from Arabidopsis seeds containing the suppression cassette hairpin+intron. The material stained in blue corresponds predominantly of protein bodies. The open circles represent oil bodies. Panel “C” shows the oil bodies from wild type like (null) Arabidopsis line segregated from plants containing the suppression cassette hairpin+intron.

FIG. 9: Thin layer chromatography of oil body-lipids. Oil bodies were isolated from different plants and total lipids were extracted from these organelles. Lipids were applied on silica Gel 60 F254 plates and half-developed with chloroform-methanol-acetic acid-formic acid-water (70:30:12:4:2 [v/v]) and fully developed with hexane-diethyl ether-acetic acid (65:35:2 [v/v]) according to Vance and Russell (1990). The lipids were visualized by heating the plates after they have been dipped in a solution containing cupric acetate (3%) and phosphoric acid (8%). Abbreviations are PC, phosphatidylcholine; PE, phosphatidylethanolamine; PS, phosphatidylserine; PI, phosphatidylinositol (PI) and TAG, triacylglycerol.

FIG. 10: The introduction of a recombinant oleosin to rescue the phenotype are showed in left panels and confocal section in right panels. (A) SupAtol1-Loop (Hairpin-Loop) plant: Left panel: SDS-PAGE profile of oil body associated proteins. Atol1 polypeptide is indicated by the black arrow. Right panel: confocal section of mature embryo. White arrows show large oil bodies. (B) MaizOl plant: Left panel: SDS-PAGE profile of oil body associated proteins. Atol1 and recombinant oleosin from Maize are indicated by the black arrows. Right panel: confocal section of mature embryo. (C) Progeny from crossing between SupAtol1-Loop (Hairpin-Loop) and MaizOl plants: Left panel: SDS-PAGE profile of oil body associated proteins. Atol1 and recombinant oleosin from Maize are indicated by the black arrows. Right panel: confocal section of mature embryo. Bars in (A) and (C)=5 μm; bar in (B)=8 μm.

FIG. 11: Comparison of germination of wild-type and SupAtol1-Loop plants in different conditions. The germination rate in each batch was scored by visualisation of radicle emergence every 24 hours. (A) Wet filter paper; Light; (B) Wet filter paper; stratified seeds; Light (C) Half strength MS media−Sucrose; Light; (D) Half strength MS media+Sucrose; Light; (E) Half strength MS media−Sucrose; Dark; (F) Half strength MS media+Sucrose; Dark.

FIG. 12: Fate of oil bodies after germination and seedling development. (A) and (B) Confocal sections of wild-type Arabidopsis seedlings after 2 and 4 days after imbibition respectively. Oilbodies were stained with Nile red. (C), (D) and (E) Confocal sections of oleosin suppressed Arabidopsis seedlings after 2 and 4 and 6 days after imbibition respectively. Oilbodies were stained with Nile red. (F) and (G) Five days old Wild-type and SupAtol1-Loop seedlings respectively germinated in half strength MS media without sucrose supplement. Bars in (A) and (B)=10 μm; bar in (C) to (E)=20 μm.

### DETAILED DESCRIPTION OF THE INVENTION

### I. Terms and Definitions

Unless defined otherwise, all technical and scientific terms used herein shall have the same meaning as is commonly understood by one skilled in the art to which the present invention belongs. Where permitted, all patents, applications, published applications, and other publications, including nucleic acid and polypeptide sequences from GenBank, SwissPro and other databases referred to in the disclosure are incorporated by reference in their entirety.

The terms “nucleic acid construct” and “nucleic acid sequence” as used herein refers to a polynucleoside or polynucleotide consisting of monomers consisting of naturally occurring bases, sugars and intersugar (backbone) linkages. The terms also include modified or substituted sequences comprising non-naturally occurring monomers or portions thereof. The nucleic acid constructs of the present invention may be deoxyribonucleic acid constructs (DNA) or ribonucleic acid constructs (e.g. RNA, mRNA) and may include naturally occurring bases including adenine, guanine, cytosine, thymidine and uracil. The constructs may also contain modified bases. Examples of such modified bases include aza and deaza adenine, guanine, cytosine, thymidine and uracil; and xanthine and hypoxanthine.

The term “chimeric” as used herein in the context of nucleic acid sequences refers to at least two linked nucleic acid sequences which are not naturally linked. For example, a nucleic acid sequence constituting a plant promoter linked to a nucleic acid sequence encoding an mRNA complementary to a nucleic acid sequence encoding an oleosin is a chimeric nucleic acid sequence.

By the term “complementary” it is meant that two nucleic acid sequences are capable of hybridizing under at least moderately stringent hybridization conditions to form a nucleic acid duplex. By the phrase “At least moderately stringent hybridization conditions” it is meant that conditions are selected which promote selective hybridization between two complementary nucleic acid molecules in solution. Hybridization may occur to all or a portion of a nucleic acid sequence molecule. The hybridizing portion is typically at least 15 (e.g. 20, 25, 30, 40 or 50) nucleotides in length. Those skilled in the art will recognize that the stability of a nucleic acid duplex, or hybrids, is determined by the Tₘ, which in sodium containing buffers is a function of the sodium ion concentration and temperature (Tₘ=81.5° C.−16.6 (Log₁₀, [Na⁺])+0.41(% (G+C)−600/1), or similar equation). Accordingly, the parameters in the wash conditions that determine hybrid stability are sodium ion concentration and temperature. In order to identify molecules that are similar, but not identical, to a known nucleic acid molecule a 1% mismatch may be assumed to result in about a 1° C. decrease in Tₘ, for example if nucleic acid molecules are sought that have a >95% identity, the final wash temperature will be reduced by about 5° C. Based on these considerations those skilled in the art will be able to readily select appropriate hybridization conditions. In preferred embodiments, stringent hybridization conditions are selected. By way of example the following conditions may be employed to achieve stringent hybridization: hybridization at 5× sodium chloride/sodium citrate (SSC)/5×Denhardt's solution/1.0% SDS at Tₘ−5° C. based on the above equation, followed by a wash of 0.2×SSC/0.1% SDS at 60° C. Moderately stringent hybridization conditions include a washing step in 3×SSC at 42° C. It is understood however that equivalent stringencies may be achieved using alternative buffers, salts and temperatures. Additional guidance regarding hybridization conditions may be found in: Current Protocols in Molecular Biology, John Wiley & Sons, N.Y., 1989, 6.3.1.-6.3.6 and in: Sambrook et al., Molecular Cloning, a Laboratory Manual, Cold Spring Harbor Laboratory Press, 1989, Vol. 3.

The term “mRNA” or “messenger RNA” as used herein refers to a polynucleotide which is the product of transcription of a DNA sequence and capable of being translated into a polypeptide.

The term “oil body” or “oil bodies” as used herein refers to any oil or fat storage organelle in plant cell (described in for example: Huang (1992) Ann. Rev. Plant Mol. Biol. 43: 177-200).

The terms “oleosin” and “oleosin polypeptides” as may be used herein interchangeably refer to any and all oleosin polypeptides, including the oleosin polypeptides listed in Table 1 (SEQ ID NO:1 to 84), as well as a polypeptide molecule which (i) is substantially identical to the amino acid sequences constituting any oleosin polypeptides set forth herein or (ii) is encoded by a nucleic acid sequence capable of hybridizing under at least moderately stringent conditions to any nucleic acid sequence encoding oleosin but for the use of synonymous codons. The oleosin polypeptide is preferably from plant origin.

By the term “substantially identical” it is meant that two polypeptide sequences preferably are at least 75% identical, and more preferably are at least 85% identical and most preferably at least 95% identical, for example 96%, 97%, 98% or 99% identical. In order to determine the percentage of identity between two polypeptide sequences the amino acid sequences of such two sequences are aligned, preferably using the Clustal W algorithm (Thompson, J D, Higgins D G, Gibson T J, 1994, Nucleic Acids Res. 22 (22): 4673-4680, together with BLOSUM 62 scoring matrix (Henikoff S. and Henikoff J. G., 1992, Proc. Natl. Acad. Sci. USA 89: 10915-10919) and a gap opening penalty of 10 and gap extension penalty of 0.1, so that the highest order match is obtained between two sequences wherein at least 50% of the total length of one of the sequences is involved in the alignment. Other methods that may be used to align sequences are the alignment method of Needleman and Wunsch (J. Mol. Biol., 1970, 48: 443), as revised by Smith and Waterman (Adv. Appl. Math., 1981, 2: 482) so that the highest order match is obtained between the two sequences and the number of identical amino acids is determined between the two sequences. Other methods to calculate the percentage identity between two amino acid sequences are generally art recognized and include, for example, those described by Carillo and Lipton (SIAM J. Applied Math., 1988, 48:1073) and those described in Computational Molecular Biology, Lesk, e.d. Oxford University Press, New York, 1988, Biocomputing: Informatics and Genomics Projects. Generally, computer programs will be employed for such calculations. Computer programs that may be used in this regard include, but are not limited to, GCG (Devereux et al., Nucleic Acids Res., 1984, 12: 387) BLASTP, BLASTN and FASTA (Altschul et al., J. Molec. Biol., 1990:215:403).

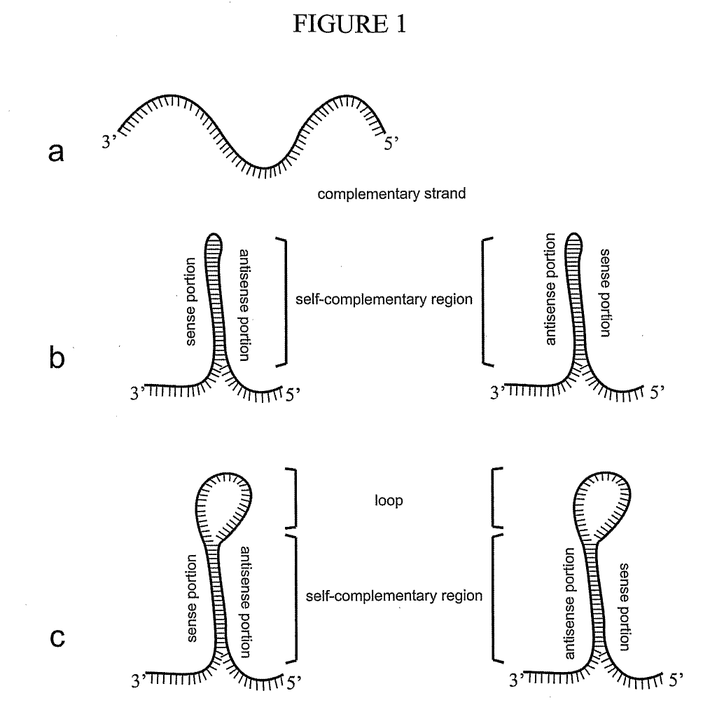

The term “hairpin” or “hairpin structure” as used herein refers to an RNA duplex structure formed by the hybridization of a first and second portion of an mRNA polynucleotide wherein the first portion of the mRNA polynucleotide is located immediately 5′ relative to the second portion of the mRNA polynucleotide (See: FIG. 1b). The “hairpin” can also further comprise 3′ and/or 5′ single-stranded region(s) extending from the double-stranded stem segment.

The term “polynucleotide loop” or “loop” as used herein refers to one or more mRNA nucleotides separating the nucleic acid sequence encoding an RNA polynucleotide complementary to a nucleic acid sequence encoding an oleosin from the nucleic acid sequence capable of encoding an oleosin (See: FIG. 1c). The polynucleotide loop can be any intervening sequence. Preferably the polynucleotide loop has no secondary structure.

The phrase “modified oleosin profile” means that the plant has steady-state oleosin levels that are reduced as compared to non-transformed plants. Preferably, seeds with a modified oleosin profile also have an increase in total protein content and a decrease in lipid content as compared to a non-transformed seed.

The “modified oleosin profile” is preferably a reduction in the steady-state levels of specific oleosin proteins as compared to the same proteins from non-transformed plants. For the purpose of this application, this reduction results after the introduction of the chimeric nucleic acid sequence into the plant cell and regeneration of a mature plant. The steady-state protein levels are reduced to a level from about 10% to about 90% compared to the unaltered protein levels. More preferably, the steady-state protein levels are reduced to a level 50% to 90% compared to the unaltered protein levels and most preferably, the steady-state protein levels are reduced 80% to 90% compared to the unaltered protein levels present in plants not comprising the chimeric nucleic acid sequence of the present invention. Techniques to determine the steady-state protein levels include densitometry, a quantitative Western blot analysis or the use of an ELISA. Examples of protocols can be found in Coligan et al. Current Protocols in Protein Science, vol 3.

### II. Preparation of Chimeric Nucleic Acid Sequences Capable of Suppressing Oleosin Gene Expression in a Plant Cell and Recombinant Expression Vectors Comprising Such Chimeric Nucleic Acid Sequences

As hereinbefore described, the present invention provides methods to suppress the expression of the endogenously present oleosin polypeptides in plants. The methods described herein are based on modifications of a plant genome with the objective of suppressing the biosynthetic production of oleosins using a chimeric nucleic acid sequence comprising a nucleic acid sequence that encodes an RNA polynucleotide complementary to a nucleic acid sequence encoding an oleosin mRNA.

Specifically, the present invention relates to preparing seed derived products from seed, in which the composition of seed storage reserves, notably the seed lipid and protein contents, have been altered. In particular the present invention provides methods for preparing seed derived products from seed, in which the seed reserves have been altered by modulation of oleosin gene expression and more particularly the suppression of oleosin gene expression.

The present inventors have found that the introduction of a nucleic acid sequence which upon transcription generates a RNA nucleic acid sequence that is complementary to an oleosin mRNA results in suppression of the expression levels of endogenous plant oleosins. This reduction in expression levels of oleosins results in a surprising modulation of the size of the plant oil bodies present in plant seeds, and, significantly, in a substantial alteration of the seed composition. In particular, using the methodologies of the present invention, the lipid and protein contents of the seed may be modulated. The methodologies herein described are further advantageous in that they permit specific modulation of the expression levels of endogenous oleosin polypeptides.

The seeds obtained in accordance with the present invention may be used to prepare a wide range of products for human and animal use, including in the formulation of food and feed products.

Accordingly, the present invention provides a method for preparing a plant seed derived product from plants seeds comprising:
(a) providing a chimeric nucleic acid construct comprising in the 5′ to 3′ direction of transcription as operably linked components:
   (i) a nucleic acid sequence capable of controlling expression in plant cells; and
   (ii) a nucleic acid sequence which upon transcription generates a RNA nucleic acid sequence that is complementary to a nucleic acid sequence encoding an oleosin mRNA or a fragment thereof;
(b) introducing the chimeric nucleic acid construct into a plant cell to obtain a transformed plant cell;
(c) regenerating from said transformed plant cell a transformed plant capable of setting seed;
(d) harvesting said seed wherein said seed has a modified oleosin profile; and
(e) preparing a plant seed derived product from said seed.

The nucleic acid sequence which upon transcription generates a RNA nucleic acid sequence that is complementary to a nucleic acid sequence encoding an oleosin mRNA that may be used in accordance the methods provided herein may be any nucleic acid sequence which upon transcription generates a RNA nucleic acid sequence that is complementary to a nucleic acid sequence encoding an oleosin mRNA or the corresponding oleosin cDNA. This sequence can be referred to as the “antisense sequence” herein. The antisense sequence complementary to a nucleic acid sequence encoding an oleosin mRNA may conveniently be prepared by selecting a DNA sequence encoding an oleosin and using such selected DNA sequence to prepare a nucleic acid sequence complementary thereto. DNA sequences encoding oleosins are well known to the art and generally available from a diverse number of sources. In accordance with the present invention DNA sequences encoding oleosins are preferably selected from a plant source. Exemplary DNA sequences that may be selected in this regard include the oleosin sequences obtainable from Arabidopsis (Van Rooijen et al (1991) Plant Mol. Bio. 18:1177-1179); maize (Qu and Huang (1990) J. Biol. Chem. Vol. 265 4:2238-2243); rapeseed (Lee and Huang (1991) Plant Physiol. 96:1395-1397); and carrot (Hatzopoulos et al (1990) Plant Cell Vol. 2, 457-467.). Oleosin sequences that may be used in accordance herewith include those set forth as SEQ ID NO:1 to SEQ ID NO:84. The corresponding nucleic acid sequences encoding the oleosin polypeptide can be readily identified via the Swiss Protein identifier numbers provided in Table 1. Using these nucleic acid sequences, additional novel oleosin encoding nucleic acid sequences may be readily identified using techniques well known to those of skill in the art. For example, libraries, such as expression libraries may be screened, and databases containing sequence information may be screened for similar sequences. In accordance herewith other methods to identify nucleic acid sequences encoding oleosins may be used and novel sequences may be discovered and used.

The nucleic acid sequence complementary to the nucleic acid sequence encoding an oleosin mRNA is preferably a DNA sequence which upon introduction in the plant cell of the chimeric nucleic acid sequence and regeneration of the plant is transcribed into a complementary RNA polynucleotide. Starting with the DNA sequence encoding an oleosin, the complementary DNA sequence may be prepared in a variety of ways including the generation of cDNA sequences using reverse transcription of mRNA. A protocol for reverse-transcriptase PCR (RT-PCR) can be found in Sambrook et al., Molecular Cloning, a Laboratory Manual, Cold Spring Harbor Laboratory Press, 1989, Vol. 3. Preferably the cDNA sequence does not contain any secondary structure. More preferably, the cDNA sequence also contains a poly-A tail for enhanced stability.

The length of the DNA sequence complementary to the nucleic acid sequence encoding an oleosin sequence may vary, provided however, that a sequence is used which upon expression of the chimeric nucleic acid sequence in the regenerated plant results in a reduction of the endogenously present levels of plant oleosins. The term “suppression” as used herein describes a reduction in the steady-state levels of specific proteins. For the purpose of this application, this reduction results after the introduction of the chimeric nucleic acid sequence into the plant cell and regeneration of a mature plant. The steady-state oleosin protein levels are reduced to a level from about 10% to about 90% compared to the unaltered protein levels. More preferably, the steady-state oleosin protein levels are reduced to a level 50% to 90% compared to the unaltered protein levels and most preferably, the steady-state oleosin protein levels are reduced 80% to 90% compared to the unaltered protein levels present in plants not comprising the chimeric nucleic acid sequence of the present invention. Techniques to determine the steady-state protein levels include densitometry, a quantitative Western blot analysis or the use of an ELISA. Examples of protocols can be found in Coligan et al. Current Protocols in Protein Science, vol 3. The techniques listed above may be performed on either a total seed extract or on the oil body fraction.

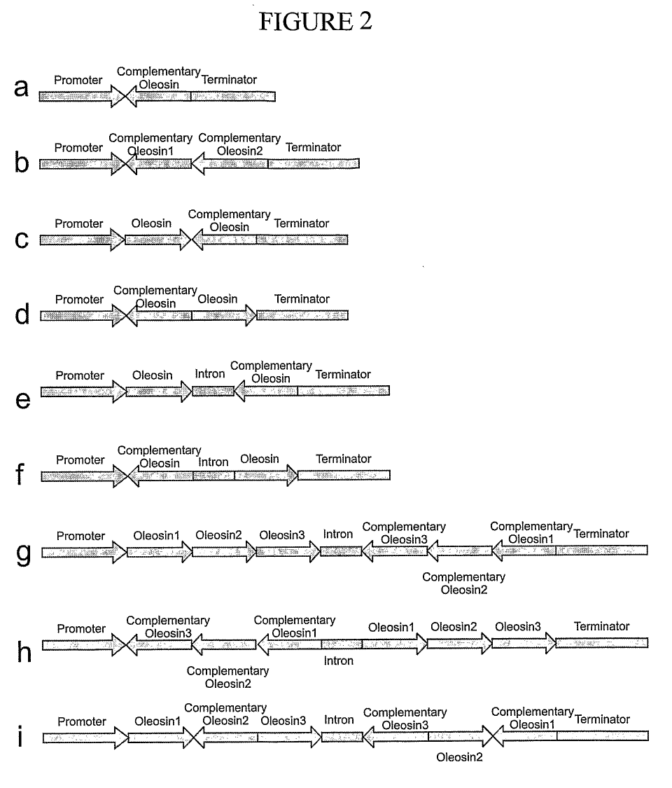

Preferably the DNA sequence complementary to the nucleic acid sequence encoding an oleosin is the same length as the DNA sequence encoding the oleosin (see FIG. 2a) and the percentage sequence identity relative to the DNA sequence complementary to the sequence encoding the oleosin is 100%, however shorter fragments, complementary to only a portion of the sequence encoding an oleosin may also be used and the percentage identity may be lower for example 99%, 98,%, 97%, 96%, 95%, 94%, 93%, 92%, 91% or 90%. Where shorter fragments are used, such fragments may for example be 95%, 90%, 85%, 80% or 75% of the length of the entire oleosin nucleotide sequence. In a preferred embodiment the nucleic acid sequence encoding a RNA polynucleotide complementary to a nucleic acid sequence encoding an oleosin mRNA is selected from SEQ ID NO:85 and 86.

In a preferred embodiment of the present invention the chimeric nucleic acid sequence further includes a nucleic acid sequence capable of encoding an oleosin. The nucleic acid encoding an oleosin can be any nucleic acid sequence that encodes an oleosin or oleosin polypeptide as defined herein. This nucleic acid sequence can also be referred to as the “sense sequence” herein.

Accordingly, the present invention provides a method to suppress expression of an oleosin protein in a plant comprising:
(a) providing a chimeric nucleic acid construct comprising in the 5′ to 3′ direction of transcription as operably linked components:
   (i) a nucleic acid sequence capable of controlling expression in plant cells;
   (ii) a nucleic acid sequence which upon transcription generates a RNA nucleic acid sequence that is complementary to a nucleic acid sequence encoding an oleosin mRNA or a fragment thereof; and
   (iii) a nucleic acid sequence encoding an oleosin or a fragment thereof, wherein said nucleic acid sequence is complementary to the nucleic acid sequence provided in (ii);
(b) introducing the chimeric nucleic acid construct into a plant cell to obtain a transformed plant cell; and
(c) regenerating from said transformed plant cell a transformed plant wherein expression of an oleosin protein is suppressed in said transformed plant.

In another preferred embodiment, the present invention provides a method for preparing a plant seed derived product from plant seeds comprising:
(a) providing a chimeric nucleic acid construct comprising in the 5′ to 3′ direction of transcription as operably linked components:
   (i) a nucleic acid sequence capable of controlling expression in plant seed cells;
   (ii) a nucleic acid sequence which upon transcription generates a RNA nucleic acid sequence that is complementary to a nucleic acid sequence encoding an oleosin mRNA sequence or fragment thereof; and
   (iii) a nucleic acid sequence encoding an oleosin or a fragment thereof, wherein said nucleic acid sequence is complementary to the nucleic acid sequence provided in (ii);
(b) introducing the chimeric nucleic acid construct into a plant cell;
(c) regenerating from said transformed plant cell a transformed plant capable of setting seed;
(d) harvesting said seed wherein said seed has a modified oleosin profile; and
(e) preparing a plant seed derived product from said seed.

It is expected that upon transcription within a transformed plant cell with the chimeric nucleic acid sequence which upon transcription generates a RNA nucleic acid sequence that is complementary to a nucleic acid sequence encoding an oleosin which in accordance herewith has been linked to a nucleic acid sequence encoding an oleosin or a fragment thereof, a single stranded mRNA is synthesized and that upon synthesis, due to the complementarity of the nucleic acid sequences (ii) and (iii) such mRNA will form a duplex structure. In a preferred embodiment, a duplex structure known as a hairpin is formed. The term “hairpin” has been defined previously herein and is shown schematically in FIG. 1b.

In a preferred embodiment, the nucleic acid sequence which upon transcription generates a RNA nucleic acid sequence that is complementary to a nucleic acid sequence encoding an oleosin is complementary to the full length oleosin nucleotide sequence and the nucleic acid sequence capable of encoding an oleosin or a fragment thereof is capable of encoding a full length oleosin (See: FIGS. 2 c and 2d).

In other embodiments, the nucleic acid sequence which upon transcription generates a RNA nucleic acid sequence that is complementary to a nucleic acid sequence encoding an oleosin is complementary to the full length oleosin nucleotide sequence, however only a fragment of a nucleic acid sequence encoding an oleosin is used. Preferably a fragment is selected which is capable of forming a hairpin. In yet other embodiments, the nucleic acid sequence encoding an oleosin is capable of encoding a full length oleosin and the RNA polynucleotide complementary to a nucleic acid sequence encoding an oleosin is complementary to only a fragment of the full length oleosin nucleotide sequence. In particularly preferred embodiments, the fragment that is used is capable of forming a hairpin. The length of such a fragment may vary but will generally be 23, 24, 25, 26, 27, 28, 29, 30 or more nucleotides in length (Thomas et al., (2001) Plant J. 25(4): 417-425). In a preferred embodiment the nucleotide sequence encoding an oleosin or a fragment thereof where in said nucleotide sequence is complementary to a nucleic acid sequence that encodes a RNA polynucleotide complementary to a nucleic acid sequence encoding an oleosin mRNA or a fragment thereof is selected from SEQ ID NO:87 and 88.

As hereinbefore mentioned a hairpin structure may be formed between the nucleic acid sequence encoding an RNA polynucleotide complementary to a nucleic acid sequence encoding an oleosin which is linked to a nucleic acid sequence encoding an oleosin. However in alternate embodiments of the present invention, the nucleic acid sequence which upon transcription generates a RNA nucleic acid sequence that is complementary to a nucleic acid sequence encoding an oleosin (the antisense sequence) is separated by one of or more nucleotides from a nucleic acid sequence capable of encoding an oleosin (the sense sequence). These separating nucleotides form a polynucleotide loop but do not generally participate in the formation of a duplex structure. The terms “polynucleotide loop” or “loop” have been defined previously herein and is shown schematically in FIG. 1c.

In a preferred embodiment, said polynucleotide loop is 1 to 150 nucleotides in length. In a further preferred embodiment, said polynucleotide loop is 50 to 100 nucleotides in length and most preferably, said loop is 70 to 80 nucleotides in length. In a preferred embodiment, said polynucleotide loop is a poly A, poly U, poly C or poly G nucleotide chain. In a preferred embodiment, said poly A, poly U, poly C or poly G nucleotide chain is 2 to 150 nucleotides in length. In a further preferred embodiment, said poly A, poly U, poly C or poly G nucleotide chain is 10 to 150 nucleotides in length. In a further preferred embodiment, said poly A, poly U, poly C or poly G nucleotide chain is 50 to 100 nucleotides in length and most preferably, said poly A, poly U, poly C or poly G nucleotide chain is 20 to 80 nucleotides in length.

In a further preferred embodiment, said polynucleotide loop comprises at least a poly A, poly U, poly C or poly G nucleotide chain wherein said poly A, poly U, poly C or poly G nucleotide chain comprises at least 2, 5, 10, 15, 20, 25, 30, 35 or 40 consecutive A, U, C or G nucleotide residues. In a further preferred embodiment, said loop comprises at least a poly A, poly U, poly C or poly G nucleotide chain wherein said poly A, poly U, poly C or poly G nucleotide chain comprises at least 5%, 10%, 15%, 20%, 25%, 30%, 35%, 40%, 45% or 50% of the total length of said polynucleotide loop.

In a further preferred embodiment, said polynucleotide loop comprises at least an (AC), (AU), (AG), (UC), (UG), (UA), (CU), (CG), (CA), (GU), (GA) or (GC) nucleotide chain wherein said (AC), (AU), (AG), (UC), (UG), (UA), (CU), (CG), (CA), (GU), (GA) or (GC) nucleotide chain comprises at least 1, 2, 5, 10, 15, 20, 25, 30, 35 or 40 consecutive (AC), (AU), (AG), (UC), (UG), (UA), (CU), (CG), (CA), (GU), (GA) or (GC) nucleotide residues. In a further preferred embodiment, said polynucleotide loop comprises at least a (AC), (AU), (AG), (UC), (UG), (UA), (CU), (CG), (CA), (GU), (GA) or (GC) nucleotide chain wherein said (AC), (AU), (AG), (UC), (UG), (UA), (CU), (CG), (CA), (GU), (GA) or (GC) nucleotide chain comprises at least 5%, 10%, 15%, 20%, 25%, 30%, 35%, 40%, 45% or 50% of the total length of said polynucleotide loop.

In a preferred embodiment the polynucleotide loop is an intron, in a further preferred embodiment the intron is a plant intron. Plant introns can vary widely in length, but approximately ⅔ of all plant introns are shorter than 150 nucleotides and the majority of introns fall in the range of 80 to 139 nucleotides in length (Simpson G G and Filipowicz W. (1996) Plant Mol Biol 32: 1-41.) The minimal functional length of an intron has been determined to be approximately 70 nucleotides in higher plants (Goodall G J and Filipowics W. (1990) Plant Mol Biol 14: 727-733.). In a further preferred embodiment the intron contains a classical splice site which consists of both a 5′ and 3′ splice site sequence. In plants the wider 5′ splice site consensus in higher plants is AG/GUAAGU. Minimally the 5′ splice site comprises the /GU dinucleotide. In rare instances, the 5′ splice site comprises a /GC dinucleotide. In plants, the wider 3′ splice site consensus in plants is UGYAG/GU. Minimally, the 3′ splice site comprises the dinucleotide, AG/. (Simpson G G and Filipowicz W. (1996) Plant Mol Biol 32: 1-41.). In a preferred embodiment the polynucleotide loop is an intron obtainable from a nucleotide sequence encoding an oleosin. In the most preferred embodiment the sequence of the polynucleotide loop is selected from SEQ ID NO:89-91.

Accordingly, the present invention provides a method to suppress expression of an oleosin protein in a plant comprising:
(a) providing a chimeric nucleic acid construct comprising in the 5′ to 3′ direction of transcription as operably linked components:
   (i) a nucleic acid sequence capable of controlling expression in plant cells;
   (ii) a nucleic acid sequence which upon transcription generates a RNA nucleic acid sequence that is complementary to a nucleic acid sequence encoding an oleosin mRNA or a fragment thereof;
   (iii) a nucleic acid sequence encoding a polynucleotide loop; and
   (iv) a nucleic acid sequence encoding an oleosin or a fragment thereof, wherein said nucleic acid sequence is complementary to the nucleic acid sequence provided in (ii);
(b) introducing the chimeric nucleic acid construct into a plant cell to obtain a transformed plant cell; and
(c) regenerating from said transformed plant cell a transformed plant wherein expression of an oleosin protein is suppressed in said transformed plant.

In a preferred embodiment, the present invention provides a method for preparing a plant seed derived product from plant seeds comprising:
(a) providing a chimeric nucleic acid construct comprising in the 5′ to 3′ direction of transcription as operably linked components:
   (i) a nucleic acid sequence capable of controlling expression in plant seed cells;
   (ii) a nucleic acid sequence which upon transcription generates a RNA nucleic acid sequence that is complementary to a nucleic acid sequence encoding an oleosin mRNA sequence or fragment thereof;
   (iii) a nucleic acid sequence encoding a polynucleotide loop; and
   (iv) a nucleic acid sequence encoding an oleosin or a fragment thereof, wherein said nucleic acid sequence is complementary to the nucleic acid sequence provided in (ii);
(b) introducing the chimeric nucleic acid construct into a plant cell;
(c) regenerating from said transformed plant cell a transformed plant capable of setting seed;
(d) harvesting said seed wherein said seed has a modified oleosin profile; and
(e) preparing a plant seed derived product from said seed.

In accordance with the present invention, the chimeric nucleic acid sequence is incorporated in a recombinant expression vector. Accordingly the present invention provides recombinant expression vectors suitable for the expression in a plant cell comprising a chimeric nucleic acid sequence comprising in the 5′ to 3′ direction of transcription:
(a) a nucleic acid sequence capable of controlling expression in a plant cell operatively linked to;
(b) a nucleic acid sequence which upon transcription generates a RNA nucleic acid sequence that is complementary to a nucleic acid sequence encoding an oleosin mRNA or a fragment thereof.

The term “suitable for expression in the selected cell” means that the recombinant expression vector contains all nucleic acid sequences required to ensure expression in the selected cell. Accordingly, the recombinant expression vectors further contain regulatory nucleic acid sequences selected on the basis of the cell which is used for expression and ensuring initiation and termination of transcription operatively linked to the nucleic acid sequence encoding the modified oleosin. Nucleic acid sequences capable of controlling expression include promoters, enhancers, silencing elements, ribosome binding sites, Shine-Dalgarno sequences, introns and other expression elements. “Operatively linked” is intended to mean that the nucleic acid sequences comprising the regulatory regions linked to the nucleic acid sequences encoding the anti-sense oleosin expression in the cell. A typical nucleic acid construct comprises in the 5′ to 3′ direction a promoter region capable of directing expression, a coding region comprising the modified oleosin polypeptide and a termination region functional in the selected cell. The selection of regulatory sequences will depend on the plant and the cell type in which the modified oleosin is expressed, and may influence the expression levels of the mRNA. Regulatory sequences are generally art-recognized and selected to direct expression of the modified oleosin in the cell.

Promoters functional in plant cells that may be used herein include constitutive promoters such as the 35S CaMV promoter (Rothstein et al., 1987 Gene 53: 153-161) the actin promoter (McElroy et al., 1990 Plant Cell 2: 163-171) and the ubiquitin promoter (European Patent Application 0 342 926). Other promoters are specific to certain tissues or organs (for example, roots, leaves, flowers or seeds) or cell types (for example, leaf epidermal cells, mesophyll cells or root cortex cells) and or to certain stages of plant development. Timing of expression may be controlled by selecting an inducible promoter, for example the PR-a promoter described in U.S. Pat. No. 5,614,395. Selection of the promoter therefore depends on the desired location and timing of the accumulation of the desired polypeptide.

In a particular preferred embodiment, the RNA polynucleotide complementary to a nucleic acid sequence encoding an oleosin mRNA or a fragment thereof expressed in a seed cell and seed specific promoters are utilized. Seed specific promoters that may be used herein include for example the phaseolin promoter (Sengupta-Gopalan et al., 1985 Proc. Natl. Acad. Sci. USA: 82 3320-3324), and the Arabidopsis 18 kDa oleosin promoter (van Rooijen et al., 1992 Plant. Mol. Biol. 18: 1177-1179). New promoters useful in various plant cell types are constantly discovered. Numerous examples of plant promoters may be found in Ohamuro et al. (Biochem of Pl., 1989 15: 1-82). In a preferred embodiment, the promoter is a constitutive promoter. Examples of constitutive promoters include, but are not limited to, 35S CaMV promoter (Rothstein et al., 1987 Gene 53: 153-161) the actin promoter (McElroy et al., 1990 Plant Cell 2: 163-171) and the ubiquitin promoter (European Patent Application 0 342 926). In a further preferred embodiment, the promoter has the precise timing and tissue specificity of the oleosin gene to be suppressed. In the most preferred embodiment, the promoter from the oleosin gene to be suppressed is used.

Genetic elements capable of enhancing expression of the polypeptide may be included in the expression vectors. In plant cells these include for example, the untranslated leader sequences from viruses such as the AMV leader sequence (Jobling and Gehrke, 1987 Nature 325: 622-625) and the intron associated with the maize ubiquitin promoter (See: U.S. Pat. No. 5,504,200).

Transcriptional terminators are generally art recognized and besides serving as a signal for transcription termination serve as a protective element serving to extend the mRNA half-life (Guarneros et al., 1982 Proc. Natl. Acad. Sci. USA 79: 238-242). In nucleic acid sequences for the expression in plant cells, the transcriptional terminator typically is from about 200 nucleotide to about 1000 nucleotides in length. Terminator sequences that may be used herein include for example, the nopaline synthase termination region (Bevan et al., 1983 Nucl. Acid. Res. 11: 369-385), the phaseolin terminator (van der Geest et al., 1994 Plant J. 6: 413-423), the terminator for the octopine synthase gene of Agrobacterium tumefaciens or other similarly functioning elements. Transcriptional terminators can be obtained as described by An (1987) Methods in Enzym. 153: 292. The selection of the transcriptional terminator may have an effect on the rate of transcription.

The recombinant expression vector further may contain a marker gene. Marker genes that may be used in accordance with the present invention include all genes that allow the distinction of transformed cells from non-transformed cells including all selectable and screenable marker genes. A marker may be a resistance marker such as an antibiotic resistance marker against for example kanamycin, ampicillin, G418, bleomycin hygromycin, chloramphenicol which allows selection of a trait by chemical means or a tolerance marker against for example a chemical agent such as the normally phytotoxic sugar mannose (Negrotto et al., 2000 Plant Cell Rep. 19: 798-803). In plant recombinant expression vectors herbicide resistance markers may conveniently be used for example markers conferring resistance against glyphosate (U.S. Pat. Nos. 4,940,935 and 5,188,642) or phosphinothricin (White et al., 1990 Nucl. Acids Res. 18: 1062; Spencer et al., 1990 Theor. Appl. Genet. 79: 625-631). Resistance markers to a herbicide when linked in close proximity to the oleosin protein may be used to maintain selection pressure on a population of plant cells or plants for those plants that have not lost the protein of interest. Screenable markers that may be employed to identify transformants through visual observation include beta-glucuronidase (GUS) (see U.S. Pat. No. 5,268,463 and U.S. Pat. No. 5,599,670) and green fluorescent protein (GFP) (Niedz et al., 1995 Plant Cell Rep. 14: 403).

Recombinant expression vectors suitable for the introduction of nucleic acid sequences in plant cells include Agrobacterium and Rhizobium based vectors such as the Ti and Ri plasmids. Agrobacterium based vectors typically carry at least one T-DNA border sequence and include vectors such pBIN 19 (Bevan, 1984 Nucl Acids Res. Vol. 12, 22:8711-8721) and other binary vector systems (for example: U.S. Pat. No. 4,940,838).

As hereinbefore mentioned, in a preferred embodiment of the present invention, the nucleic acid sequence capable of controlling expression in plant cells permits expression in plant seed cells and the transformed plant is a plant capable of setting seed. In a further preferred embodiment, the promoter is a seed-preferred promoter. Accordingly the present invention provides a method to suppress expression of an oleosin protein in the seed of a plant comprising:
(a) providing a chimeric nucleic acid construct comprising in the 5′ to 3′ direction of transcription as operably linked components:
   (i) a nucleic acid sequence capable of controlling expression in plant seed cells; and
   (ii) a nucleic acid sequence which upon transcription generates a RNA nucleic acid sequence that is complementary to a nucleic acid sequence encoding an oleosin mRNA or a fragment thereof;
(b) introducing the chimeric nucleic acid construct into a plant cell to obtain a transformed plant cell;
(c) regenerating from said transformed plant cell a transformed plant; and
(d) growing the transformed plant into a mature plant capable of setting seed, wherein oleosin expression levels are suppressed in the seed.

The recombinant expression vectors and chimeric nucleic acid sequences of the present invention may be prepared in accordance with methodologies well known to those skilled in the art of molecular biology. Such preparation will typically involve the bacterial species Escherichia coli as an intermediary cloning host. The preparation of the E. coli vectors as well as the plant transformation vectors may be accomplished using commonly known techniques such as restriction digestion, ligation, gel ectrophoresis, DNA sequencing, the Polymerase Chain Reaction (PCR) and other methodologies. A wide variety of cloning vectors is available to perform the necessary steps required to prepare a recombinant expression vector. Among the vectors with a replication system functional in E. coli, are vectors such as pBR322, the pUC series of vectors, the M13 mp series of vectors, pBluescript etc. Typically, these cloning vectors contain a marker allowing selection of transformed cells. Nucleic acid sequences may be introduced in these vectors, and the vectors may be introduced in E. coli grown in an appropriate medium. Recombinant expression vectors may readily be recovered from cells upon harvesting and lysing of the cells. Further, general guidance with respect to the preparation of recombinant vectors may be found in, for example: Sambrook et al., Molecular Cloning, a Laboratory Manual, Cold Spring Harbor Laboratory Press, 1989, Vol. 3.

### III. Preparation of Plants in which the Endogenous Oleosin Levels are Suppressed.

In accordance with the present invention, the recombinant expression vectors are introduced into the cell that is selected and the selected cells are grown to produce the modified oleosin protein in a progeny cell.

Methodologies to introduce recombinant expression vectors into a cell also referred to herein as “transformation” are well known to the art and vary depending on the cell type that is selected. General techniques to transfer the recombinant expression vectors into the cell include electroporation; chemically mediated techniques, for example CaCl₂ mediated nucleic acid uptake; particle bombardment (biolistics); the use of naturally infective nucleic acid sequences for example virally derived nucleic acid sequences or when plant cells are used Agrobacterium or Rhizobium derived nucleic acid sequences; PEG mediated nucleic acid uptake, microinjection, and the use of silicone carbide whiskers (Kaeppler et al., 1990 Plant Cell Rep. 9:415-418) all of which may be used herein.

Introduction of the recombinant expression vector into the cell may result in integration of its whole or partial uptake into host cell genome including the chromosomal DNA or the plastid genome. In a preferred embodiment the chimeric nucleic acid construct is introduced into the plant cell under nuclear genomic integration conditions. Under such conditions the chimeric nucleic acid sequence is stably integrated in the plant's genome. Alternatively the recombinant expression vector may not be integrated into the genome and replicate independently of the host cell's genomic DNA. Genomic integration of the nucleic acid sequence is typically used as it will allow for stable inheritance of the introduced nucleic acid sequences by subsequent generations of cells and the creation, plant lines.

Particular embodiments involve the use of plant cells. Particular plant cells used herein include cells obtainable from Arabidopsis thaliana, Brazil nut (Betholletia excelsa); castor (Riccinus coinnunis); coconut (Cocus nucifera); coriander (Coriandrum sativum); cotton (Gossypium spp.); groundnut (Arachis hypogaea); jojoba (Simmondsia chinensis); linseed/flax (Linum usitatissimum); maize (Zea mays); mustard (Brassica spp. and Sinapis alba); oil palm (Elaeis guineeis); olive (Olea europaea); rapeseed (Brassica spp.); safflower (Carthamus tinctorius); soybean (Glycine max); squash (Cucurbita maxima); barley (Hordeum vulgare); wheat (Traeticum aestivum) and sunflower (Helianthus annuus).

Transformation methodologies for dicotelydenous plant species are well known. Generally Agrobacterium mediated transformation is utilized because of its high efficiency as well as the general susceptibility by many, if not all dicotelydenous plant species. Agrobacterium transformation generally involves the transfer of a binary vector (e.g. pBIN19) comprising the DNA of interest to an appropriate Agrobacterium strain (e.g. CIB542) by for example tri-parental mating with an E. coli strain carrying the recombinant binary vector and an E. coli strain carrying a helper plasmid capable of mobilization of the binary vector to the target Agrobacterium strain, or by DNA transformation of the Agrobacterium strain (Hofgen et al. Nucl. Acids. Res., 1988 16: 9877. Other transformation methodologies that may be used to transform dicotelydenous plant species include biolistics (Sanford, 1988 Trends in Biotechn. 6: 299-302); electroporation (Fromm et al., 1985 Proc. Natl. Acad. Sci. USA 82: 5824-5828); PEG mediated DNA uptake (Potrykus et al., 1985 Mol. Gen. Genetics 199: 169-177); microinjection (Reich et al., 1986 Bio/Techn. 4: 1001-1004) and silicone carbide whiskers (Kaeppler et al., 1990 Plant Cell Rep. 9: 415-418). The exact transformation methodologies typically vary somewhat depending on the plant species that is used.

In a particular embodiment Arabidopsis, safflower, or flax plant cells are used. Safflower transformation has been described by Baker and Dyer (1996 Plant Cell Rep. 16: 106-110.) Flax transformation has been described by Dong J. and McHughen A. (Plant Cell Reports (1991) 10:555-560), Dong J. and McHughen A. (Plant Sciences (1993) 88:61-71) and Mlynarova et al. (Plant Cell Reports (1994) 13: 282-285). Additional plant species specific transformation protocols may be found in: Biotechnology in Agriculture and Forestry 46: Transgenic Crops I (Y.P.S. Bajaj ed.), Springer-Verlag, New York (1999), and Biotechnology in Agriculture and Forestry 47: Transgenic Crops II (Y.P.S. Bajaj ed.), Springer-Verlag, New York (2001).

Monocotyledonous plant species may be transformed using a variety of methodologies including particle bombardment (Christou et al., 1991 Biotechn. 9: 957-962; Weeks et al., 1993 Plant Physiol. 102: 1077-1084; Gordon-Kamm et al., 1990 Plant Cell 2: 603-618) PEG mediated DNA uptake (EP 0 292 435; 0 392 225) or Agrobacterium-mediated transformation (Goto-Fumiyuki et al., 1999 Nature-Biotech. 17 (3):282-286).

Plastid transformation is described in U.S. Pat. Nos. 5,451,513; 5,545,817 and 5,545,818; and PCT Patent Applications 95/16783; 98/11235 and 00/39313. Basic chloroplast transformation involves the introduction of cloned plastid DNA flanking a selectable marker together with the nucleic acid sequence of interest into a suitable target tissue using for example biolistics or protoplast transformation. Selectable markers that may be used include for example the bacterial aadA gene (Svab et al., 1993 Proc. Natl. Acad. Sci. USA 90: 913-917). Plastid promoters that may be used include for example the tobacco clpP gene promoter (PCT Patent Application 97/06250).

In another embodiment, the invention chimeric nucleic acid contructs provided herein are directly transformed into the plastid genome. Plastid transformation technology is described extensively in U.S. Pat. Nos. 5,451,513, 5,545,817, 5,545,818 and 5,576,198; in PCT application nos. WO 95/16783 and WO 97/32977; and in McBride et. al., 1994 Proc Natl Acad Sci USA 91: 7301-7305, the entire disclosures of all of which are hereby incorporated by reference. In one embodiment, plastid transformation is achieved via biolistics, first carried out in the unicellular green alga Chlamydomonas reinhardtii (Boynton et al., 1988 Science 240:1534-1537)) and then extended to Nicotiana tabacum (Svab et al., 1990 Proc Natl Acad Sci USA 87:8526-8530), combined with selection for cis-acting antibiotic resistance loci (spectinomycin or streptomycin resistance) or complementation of non-photosynthetic mutant phenotypes.

In another embodiment, tobacco plastid transformation is carried out by particle bombardment of leaf or callus tissue, or polyethylene glycol (PEG)-mediated uptake of plasmid DNA by protoplasts, using cloned plastid DNA flanking a selectable antibiotic resistance marker. For example, 1 to 1.5 kb flanking regions, termed targeting sequences, facilitate homologous recombination with the plastid genome and allow the replacement or modification of specific regions of the 156 kb tobacco plastid genome. In one embodiment, point mutations in the plastid 16S rDNA and rps12 genes conferring resistance to spectinomycin and/or streptomycin can be utilized as selectable markers for transformation (Svab et al., 1990 Proc Natl Acad Sci USA 87:8526-8530; Staub et al., 1992 Plant Cell 4:39-45 the entire disclosures of which are hereby incorporated by reference), resulting in stable homoplasmic transformants at a frequency of approximately one per 100 bombardments of target leaves. The presence of cloning sites between these markers allows creation of a plastid targeting vector for introduction of foreign genes (Staub et al., 1993 EMBO J. 12:601-606, the entire disclosure of which is hereby incorporated by reference). In another embodiment, substantial increases in transformation frequency can be obtained by replacement of the recessive rRNA or r-protein antibiotic resistance genes with a dominant selectable marker, the bacterial aadA gene encoding the spectinomycin-detoxifying enzyme aminoglycoside-3′-adenyltransferase (Svab et al., 1993 Proc Natl Acad Sci USA 90: 913-917, the entire disclosure of which is hereby incorporated by reference). This marker has also been used successfully for high-frequency transformation of the plastid genome of the green alga Chlamydomonas reinhardtii (Goldschmidt-Clermont, M., 1991 Nucl Acids Res 19, 4083-4089, the entire disclosure of which is hereby incorporated by reference). In other embodiments, plastid transformation of protoplasts from tobacco and the moss Physcomitrella can be attained using PEG-mediated DNA uptake (O'Neill et al., 1993 Plant J 3:729-738; Koop et al., 1996 Planta 199:193-201, the entire disclosures of which are hereby incorporated by reference).

Both particle bombardment and protoplast transformation are also contemplated for use herein. Plastid transformation of oilseed plants has been successfully carried out in the genera Arabidopsis and Brassica (Sikdar et al., 1998 Plant Cell Rep 18:20-24; PCT Application WO 00/39313, the entire disclosures of which are hereby incorporated by reference).

A chimeric nucleic sequence construct is inserted into a plastid expression cassette including a promoter capable of expressing the construct in plant plastids. A particular promoter capable of expression in a plant plastid is, for example, a promoter isolated from the 5′ flanking region upstream of the coding region of a plastid gene, which may come from the same or a different species, and the native product of which is typically found in a majority of plastid types including those present in non-green tissues. Gene expression in plastids differs from nuclear gene expression and is related to gene expression in prokaryotes (Stern et al., 1997 Trends in Plant Sci 2:308-315, the entire disclosure of which is hereby incorporated by reference).

Plastid promoters generally contain the −35 and −10 elements typical of prokaryotic promoters, and some plastid promoters called PEP (plastid-encoded RNA polymerase) promoters are recognized by an E. coli-like RNA polymerase mostly encoded in the plastid genome, while other plastid promoters called NEP promoters are recognized by a nuclear-encoded RNA polymerase. Both types of plastid promoters are suitable for use herein. Examples of plastid promoters include promoters of clpP genes such as the tobacco clpP gene promoter (WO 97/06250, the entire disclosure of which is hereby incorporated by reference) and the Arabidopsis clpP gene promoter (U.S. application Ser. No. 09/038,878, the entire disclosure of which is hereby incorporated by reference). Another promoter capable of driving expression of a chimeric nucleic acid construct in plant plastids comes from the regulatory region of the plastid 16S ribosomal RNA operon (Harris et al., 1994 Microbiol Rev 58:700-754; Shinozaki et al., 1986 EMBO J. 5:2043-2049, the entire disclosures of both of which are hereby incorporated by reference). Other examples of promoters capable of driving expression of a nucleic acid construct in plant plastids include a psbA promoter or am rbcL promoter. A plastid expression cassette preferably further includes a plastid gene 3′ untranslated sequence (3′ UTR) operatively linked to a chimeric nucleic acid construct of the present invention. The role of untranslated sequences is preferably to direct the 3′ processing of the transcribed RNA rather than termination of transcription. An exemplary 3′ UTR is a plastid rps16 gene 3′ untranslated sequence, or the Arabidopsis plastid psbA gene 3′ untranslated sequence. In a further embodiment, a plastid expression cassette includes a poly-G tract instead of a 3′ untranslated sequence. A plastid expression cassette also preferably further includes a 5′ untranslated sequence (5′ UTR) functional in plant plastids, operatively linked to a chimeric nucleic acid construct provided herein.

A plastid expression cassette is contained in a plastid transformation vector, which preferably further includes flanking regions for integration into the plastid genome by homologous recombination. The plastid transformation vector may optionally include at least one plastid origin of replication. The present invention also encompasses a plant plastid transformed with such a plastid transformation vector, wherein the chimeric nucleic acid construct is expressible in the plant plastid. Also encompassed herein is a plant or plant cell, including the progeny thereof, including this plant plastid. In a particular embodiment, the plant or plant cell, including the progeny thereof, is homoplasmic for transgenic plastids.

Other promoters capable of driving expression of a chimeric nucleic acid construct in plant plastids include transactivator-regulated promoters, preferably heterologous with respect to the plant or to the subcellular organelle or component of the plant cell in which expression is effected. In these cases, the DNA molecule encoding the transactivator is inserted into an appropriate nuclear expression cassette which is transformed into the plant nuclear DNA. The transactivator is targeted to plastids using a plastid transit peptide. The transactivator and the transactivator-driven DNA molecule are brought together either by crossing a selected plastid-transformed line with and a transgenic line containing a DNA molecule encoding the transactivator supplemented with a plastid-targeting sequence and operably linked to a nuclear promoter, or by directly transforming a plastid transformation vector containing the desired DNA molecule into a transgenic line containing a chimeric nucleic acid construct encoding the transactivator supplemented with a plastid-targeting sequence operably linked to a nuclear promoter. If the nuclear promoter is an inducible promoter, in particular a chemically inducible embodiment, expression of the chimeric nucleic acid construct in the plastids of plants is activated by foliar application of a chemical inducer. Such an inducible transactivator-mediated plastid expression system is preferably tightly regulatable, with no detectable expression prior to induction and exceptionally high expression and accumulation of protein following induction.

A particular transactivator is, for example, viral RNA polymerase. Particular promoters of this type are promoters recognized by a single sub-unit RNA polymerase, such as the T7 gene 10 promoter, which is recognized by the bacteriophage T7 DNA-dependent RNA polymerase. The gene encoding the T7 polymerase is preferably transformed into the nuclear genome and the T7 polymerase is targeted to the plastids using a plastid transit peptide. Promoters suitable for nuclear expression of a gene, for example a gene encoding a viral RNA polymerase such as the T7 polymerase, are described above and elsewhere in this application. Expression of chimeric nucleic acid constructs in plastids can be constitutive or can be inducible, and such plastid expression can be also organ- or tissue-specific. Examples of various expression systems are extensively described in WO 98/11235, the entire disclosure of which is hereby incorporated by reference. Thus, in one aspect, the present invention utilizes coupled expression in the nuclear genome of a chloroplast-targeted phage T7 RNA polymerase under the control of the chemically inducible PR-1a promoter, for example of the PR-1 promoter of tobacco, operably linked with a chloroplast reporter transgene regulated by T7 gene 10 promoter/terminator sequences, for example as described in as in U.S. Pat. No. 5,614,395 the entire disclosure of which is hereby incorporated by reference. In another embodiment, when plastid transformants homoplasmic for the maternally inherited TR or NTR genes are pollinated by lines expressing the T7 polymerase in the nucleus, F1 plants are obtained that carry both transgene constructs but do not express them until synthesis of large amounts of enzymatically active protein in the plastids is triggered by foliar application of the PR-1a inducer compound benzo(1,2,3)thiadiazole-7-carbothioic acid S-methyl ester (BTH).

Following transformation the cells are grown, typically in a selective medium allowing the identification of transformants. Cells may be harvested in accordance with methodologies known to the art. These methodologies are generally cell-type dependent and known to the skilled artisan. Where plants are employed they may be regenerated into mature plants using plant tissue culture techniques generally known to the skilled artisan. Seeds may be harvested from mature transformed plants and used to propagate the plant line. Plants may also be crossed and in this manner, contemplated herein is the breeding of cells lines and transgenic plants that vary in genetic background.

It should be noted that, plant genomes may comprise one or more nucleotide sequences encoding an oleosin, typically each varying somewhat in nucleic acid sequence. It may be desirable to simultaneously suppress the expression of a plurality of oleosins. Accordingly a plurality of chimeric sequences may be prepared, each designed to suppress expression of a different oleosin nucleotide sequence. In accordance herewith separate vectors comprising such chimeric nucleic acid sequences may simulataneously be introduced into a plant cell. Alternatively a single vector comprising a plurality of chimeric nucleic acid sequences, each chimeric sequence comprising (i) a nucleic acid sequence capable of directing transcription in a plant cell; (ii) a nucleic acid sequence which upon transcription generates a RNA nucleic acid sequence that is complementary to a nucleic acid sequence encoding an oleosin and (iii) a nucleic acid sequence encoding an oleosin may be introduced into a plant cell (see FIG. 2b, g, h, i). Alternatively, in order to achieve suppression multiple oleosins, upon having prepared a plant using one chimeric nucleic acid sequence in accordance herewith, such a plant may be transformed with one or more additional chimeric nucleic acid sequences each targeting a different endogenous plant oleosin.

In one aspect the present invention also provides plants and plant seeds in which oleosin gene expression has been suppressed comprising in the 5′ to 3′ direction of transcription as operably linked components:
(i) a nucleic acid sequence capable of controlling expression in plant cells; and
(ii) a nucleic acid sequence which upon transcription generates a RNA nucleic acid sequence that is complementary to a nucleic acid sequence encoding an oleosin mRNA or a fragment thereof.

In a further aspect, the present invention also includes oil bodies comprising a modified oleosin profile of the invention.

### IV. Uses of Plant Seeds Comprising Suppressed Levels of Oleosin

As hereinbefore mentioned the seed obtained in accordance with the present invention may be used to prepare a seed derived product. Seed derived products that may be prepared in accordance with the present invention include products for human and animal use, including food and feed products and personal care products.

One of skill in the art can readily determine how to prepare the plant seed derived product which will depend on the nature of the product. For example, the plant seed derived product may be prepared using any standard commercial processing practices for seed. Whole seeds or crushed seeds may be used to prepare the seed derived products, for example food products. Alternatively seed fractions are prepared which then are used to prepare the seed derived product. Preferred methods that may be used in accordance herewith include solvent extraction, such as extraction by hexane and the application of mechanical force, for example pressing, grinding or milling. Typically, these processes result in the separation of the seed oil fraction from the protein, also termed the meal, fraction. The isolated meal and oil fraction may both be used for further processing in food or feed products or personal care products.

Seed derived products for human consumption that may be prepared in accordance with the present invention include any food product including any health food that is capable of imparting health benefits. Beverages that may be prepared from seed products prepared in accordance with the present invention include any beverage in dry powdered or liquid form, for example any fruit juice, fresh frozen or canned concentrate, flavored drinks as well as adult and infant formulas. Further products include products prepared from a non-dairy milk, such as soy milk. These products include whole milk, skim milk, ice cream, yoghurt and the like.

Animal feed products that may be prepared using seed prepared in accordance with the present invention include products intended feed products, including products for use in fish and shrimp aquaculture, and pet food products, intended for feeding to dogs, cats, birds, reptiles, rodents and the like.

Personal care products that may be prepared using a seed derived product prepared in accordance with the present invention include any cosmetic product for human use, including soaps, skin creams facial creams, face masks, skin cleanser, tooth paste, lipstick, perfumes, make-up, foundation, blusher, mascara, eyeshadow, sunscreen lotions, hair conditioner, and hair colouring.

The exact seed processing conditions as well as the plant seed derived product preparation methodology, employed will vary depending on the plant species as well as on the desired plant seed derived product the seed is processed into. The exact processing conditions of the seed or the preparation techniques for the seed derived employed are considered to be immaterial to the present invention.

### Modulating the Nutritional Value of Seed

As hereinbefore mentioned, the present invention provides a method to alter the composition of plants, notably plant seeds. In particular, in accordance with the present invention seeds may be prepared in which the lipid content is reduced, whereas the protein content within the seed is increased relative to seed obtained from wild type plant seeds.

Accordingly, the present invention also provides a method to increase the protein content in plants seeds comprising:
(a) providing a chimeric nucleic acid construct comprising in the 5′ to 3′ direction of transcription as operably linked components:
   (i) a nucleic acid sequence capable of controlling expression in plant cells; and
   (ii) a nucleic acid sequence which upon transcription generates a RNA nucleic acid sequence that is complementary to a nucleic acid sequence encoding an oleosin mRNA or a fragment thereof;
(b) introducing the chimeric nucleic acid construct into a plant cell to obtain a transformed plant cell;
(c) regenerating from said transformed plant cell a transformed plant capable of setting seed; and
(d) obtaining the seed wherein the seed has an increase in the total protein content of the seed as compared to a non-transformed plant.

Accordingly, the present invention also provides a method to decrease the lipid content in plants seeds comprising:
(a) providing a chimeric nucleic acid construct comprising in the 5′ to 3′ direction of transcription as operably linked components:
   (i) a nucleic acid sequence capable of controlling expression in plant cells; and
   (ii) a nucleic acid sequence which upon transcription generates a RNA nucleic acid sequence that is complementary to a nucleic acid sequence encoding an oleosin mRNA or a fragment thereof;
(b) introducing the chimeric nucleic acid construct into a plant cell to obtain a transformed plant cell;
(c) regenerating from said transformed plant cell a transformed plant capable of setting seed; and
(d) obtaining the seed wherein the seed has a decrease in the total lipid content of the seed as compared to a non-transformed plant.

The seeds thus obtained may be used to prepare a plant seed derived product.

The plant seeds with a modified oleosin protein profile within the seed have an increase in the total protein content of said plants seeds. For the purpose of this application, this increase in protein results after the introduction of the chimeric nucleic acid sequence into the plant cell and regeneration of a mature plant. The increase in total protein content of the plant seeds with a modified oleosin profile is increased to a level from about 5% to about 30% relative to the total protein content of the plant seeds from wild type plants with unaltered protein levels. More preferably, the total protein content of the plant seeds with a modified oleosin profile is increased a level 15% to 30% relative to the total protein content of the plant seeds from wild type plants with unaltered protein levels and most preferably, the total protein content of the plant seeds with a modified oleosin profile is increased to a level 20% to 30% compared to the total protein content of the plant seeds from wild type plants not comprising the chimeric nucleic acid sequence of the present invention. Techniques to determine the total protein content of plant seeds include using BCA protein assay reagent (Pierce, Rockford, Ill.) and further described in Example 5 of the present application. The techniques listed above may be performed on either a total seed extract or on the oil body fraction.

The plant seeds with a modified oleosin protein profile within the seed have a decrease in the lipid content in said plants seeds. For the purpose of this application, this decrease in lipid content results after the introduction of the chimeric nucleic acid sequence into the plant cell and regeneration of a mature plant. The decrease in lipid content of the plant seeds with a modified oleosin profile is decreased to a level from about 1% to about 20% relative to the lipid content of the plant seeds from wild type plants with unaltered protein levels. More preferably, the lipid content of the plant seeds with a modified oleosin profile is decreased a level 10% to 20% relative to the lipid content of the plant seeds from wild type plants with unaltered protein levels and most preferably, the lipid content of the plant seeds with a modified oleosin profile is increased to a level 15% to 20% compared to the lipid content of the plant seeds from wild type plants not comprising the chimeric nucleic acid sequence of the present invention. Techniques to determine the lipid content of plant seeds include are described in Bligh and Dyer (1959. Can. J. Med. Sci. 37:911-917) and further described in Example 5 of the present application.

### Oil Body Based Products

One seed fraction that may be obtained in accordance with the present invention in order to prepare a seed derived product is the oil body fraction. Accordingly, in another aspect of the present invention, the oil body fraction may be obtained using for example methods as disclosed in PCT 98/53698 and the oil body fraction may be used to prepare food, feed or personal care products.

In a preferred embodiment of the present invention provides a composition comprising oil bodies with a modified oleosin profile isolated from plant seeds. Accordingly, the present invention provides a composition comprising oil bodies with a modified oleosin profile isolated from plant seeds. The oil bodies with a modified oleosin profile are preferably prepared by a process comprising:
(a) providing a chimeric nucleic acid construct comprising in the 5′ to 3′ direction of transcription as operably linked components:
   (i) a nucleic acid sequence capable of controlling expression in plant cells;
   (ii) a nucleic acid sequence which upon transcription generates a RNA nucleic acid sequence that is complementary to a nucleic acid sequence encoding an oleosin mRNA or a fragment thereof; and
   (iii) a nucleic acid sequence encoding an oleosin or a fragment thereof, wherein said nucleic acid sequence is complementary to the nucleic acid sequence provided in (ii);
(b) introducing the chimeric nucleic acid construct into a plant cell to obtain a transformed plant cell;
(c) regenerating from said transformed plant cell a transformed plant wherein expression of an oleosin protein is suppressed in said transformed plant; and
(d) harvesting said seed and isolating oil bodies with a modified oleosin profile from said seeds.

In a further preferred embodiment of the present invention the composition comprising oil bodies isolated from plant seeds with a modified oleosin profile is prepared by a process comprising:
(a) providing a chimeric nucleic acid construct comprising in the 5′ to 3′ direction of transcription as operably linked components:
   (i) a nucleic acid sequence capable of controlling expression in plant cells;
   (ii) a nucleic acid sequence which upon transcription generates a RNA nucleic acid sequence that is complementary to a nucleic acid sequence encoding an oleosin mRNA or a fragment thereof;
   (iii) a nucleic acid sequence encoding a polynucleotide loop; and
   (iv) a nucleic acid sequence encoding an oleosin or a fragment thereof, wherein said nucleic acid sequence is complementary to the nucleic acid sequence provided in (ii);
(b) introducing the chimeric nucleic acid construct into a plant cell to obtain a transformed plant cell;
(c) regenerating from said transformed plant cell a transformed plant wherein expression of an oleosin protein is suppressed in said transformed plant; and
(d) harvesting said seed and isolating oil bodies from said seeds with a modified oleosin profile.

In order to prepare such oil bodies from plant seeds, plants are grown and allowed to set seed in accordance with common agricultural practices. Upon harvesting the seed and, if necessary the removal of large insoluble materials such as stones or seed hulls, by for example sieving or rinsing, any process suitable for the isolation of oil bodies from seeds may be used herein. A typical process involves grinding of the seeds followed by an aqueous extraction process.

Seed grinding may be accomplished by any comminuting process resulting in a substantial disruption of the seed cell membrane and cell walls without compromising the structural integrity of the oil bodies present in the seed cell. Suitable grinding processes in this regard include mechanical pressing and milling of the seed. Wet milling processes such as described for cotton (Lawhon et al., 1977 J. Am. Oil Chem. Soc. 63: 533-534) and soybean (U.S. Pat. No. 3,971,856; Carter et al., 1974 J. Am. Oil Chem. Soc. 51: 137-141) are particularly useful in this regard. Suitable milling equipment capable of industrial scale seed milling include colloid mills, disc mills, pin mills, orbital mills, IKA mills and industrial scale homogenizers. The selection of the milling equipment will depend on the seed, which is selected, as well as the throughput requirement.

Solid contaminants such as seed hulls, fibrous materials, undissolved carbohydrates, proteins and other insoluble contaminants are subsequently preferably removed from the ground seed fraction using size exclusion based methodologies such as filtering or gravitational based methods such as a centrifugation based separation process. Centrifugation may be accomplished using for example a decantation centrifuge such as a HASCO 200 2-phase decantation centrifuge or an NX310B (Alpha Laval). Operating conditions are selected such that a substantial portion of the insoluble contaminants and sediments and may be separated from the soluble fraction.

Following the removal of insolubles the oil body fraction may be separated from the aqueous fraction. Gravitational based methods as well as size exclusion based technologies may be used. Gravitational based methods that may be used include centrifugation using for example a tubular bowl centrifuge such as a Sharples AS-16 or AS-46 (Alpha Laval), a disc stack centrifuge or a hydrocyclone, or separation of the phases under natural gravitation. Size exclusion methodologies that may be used include membrane ultra filtration and crossflow microfiltration.

Separation of solids and separation of the oil body phase from the aqueous phase may also be carried out concomitantly using gravity based separation methods or size exclusion based methods.

The oil body preparations obtained at this stage in the process are generally relatively crude and depending on the application of the oil bodies, it may be desirable to remove additional contaminants. Any process capable of removing additional seed contaminants may be used in this regard. Conveniently the removal of these contaminants from the oil body preparation may be accomplished by resuspending the oil body preparation in an aqueous phase and re-centrifuging the resuspended fraction. The resuspension conditions selected may vary depending on the desired purity of the oil body fractions. The oil bodies may be resuspended one or more times depending on the desired purity and the ionic strength, pH and temperature may all be varied. Analytical techniques may be used to monitor the removal of contaminants. For example SDS gel electrophoresis may be employed to monitor the removal of seed proteins.

The entire oil body isolation process may be performed in a batch wise fashion or continuous flow. In a particular embodiment, industrial scale continuous flow processes are utilized.

Through the application of these and similar techniques the skilled artisan is able to obtain oil bodies from any cell comprising oil bodies. The skilled artisan will recognize that generally the process will vary somewhat depending on the cell type that is selected. However, such variations may be made without departing from the scope and spirit of the present invention.

The oil bodies isolated from a plant with a modified oleosin profile are larger then oil bodies found in the wild type plant. For the purpose of this application, this increase in size results after the introduction of the chimeric nucleic acid sequence into the plant cell and regeneration of a mature plant. The size of the oil bodies with a modified oleosin profile are increased to a level from about 1, 2, 3, 4, 5, 6, 7, 8, 9 to about 10 times compared to the oil bodies from wild type plants with unaltered protein levels. Preferably, the size of the oil bodies with a modified oleosin profile is increased to a level that is 2 times, more preferably 5 to 10 times, compared to the oil bodies from wild type plants with unaltered protein levels. Most preferably, the size of the oil bodies with a modified oleosin profile is increased to a level 8 to 10 times compared to the oil bodies from wild type plants not comprising the chimeric nucleic acid sequence of the present invention. Techniques to determine the size of the oil bodies in vivo include confocal microscopy. This technique allows the examination of whole mount, embryo sections and provides a more accurate size comparison between Arabidopsis lines. Embryos can be isolated from mature seeds and neutral lipids stained with Nile Red. Triacylglycerols represent the vast majority of neutral lipids in most oil seeds hence oil bodies are selectively stained by Nile Red. Examples of protocols can be found in Paddock et al. Methods in Molecular Biology, vol 122. “Confocal Microscopy—Methods and Protocols”. Techniques to determine the size of the oil bodies in vitro include using bright field conventional microscopy. Examples of protocols can be found in Bright-field, phase and dark-field microscopy., Spencer, M., Fundamentals of Light Microscopy, Cambridge University Press, New York, 32-39 (1982).

The plant seeds with a modified oleosin protein profile within the seed have decrease in the phospholipid accumulation in said plants seeds. For the purpose of this application, this decrease in phospholipid accumulation results after the introduction of the chimeric nucleic acid sequence into the plant cell and regeneration of a mature plant. The decrease in phospholipid accumulation of the plant seeds with a modified oleosin profile is decreased to a level from about 5% to about 40% relative to the phospholipid accumulation of the plant seeds from wild type plants with unaltered protein levels. More preferably, the phospholipid accumulation of the plant seeds with a modified oleosin profile is decreased a level 20% to 40% relative to the phospholipid accumulation of the plant seeds from wild type plants with unaltered protein levels and most preferably, the phospholipid accumulation of the plant seeds with a modified oleosin profile is increased to a level 30% to 40% compared to the phospholipid accumulation of the plant seeds from wild type plants not comprising the chimeric nucleic acid sequence of the present invention. Techniques to determine the phospholipid accumulation of plant seeds include are described in Vance and Russel, 1990 (J. Lipid Res 31:1491-1501.) and further described in Example 6 of the present application.

### Modulation of the Oleosin Constituents

The present invention further provides a method to modulate the oleosin constituents in a plant seed. In certain instances it may be desirable to alter the oleosin constituents of a particular plant. Thus upon transformation and regeneration of a plant in accordance herewith, a nucleic acid sequence encoding an oleosin may be introduced in such a plant line. The nucleic acid sequence encoding such an oleosin may be obtained from a different plant species.

### Increasing the Amount of Recombinant Proteins on Oil Bodies

The present invention further describes a method to increase accumulation of recombinant proteins in the surface of oil bodies. The method is based on the suppression of endogenous oleosins with concomitant expression of a recombinant oleosin. The modified oil bodies may contain higher amounts of recombinant oleosins. In a further preferred embodiment, said recombinant oleosin is covalently linked to a second recombinant protein to form a chimeric protein as disclosed in WO 93/21320 and related applications which are incorporated by reference in its entirety. The use of a recombinant oleosin protein as a carrier or targeting means provides a simple mechanism to recover proteins. The chimeric protein associated with the oil body may be separated away from the bulk of cellular components in a single step by isolation of the oil body fraction using for example centrifugation size exclusion or floatation. The invention contemplates the use of heterologous proteins, including enzymes, therapeutic proteins, diagnostic proteins and the like fused to a recombinant oleosin and associated with oil bodies. Association of the protein with the oil body allows subsequent recovery of the protein by simple means (centrifugation and floatation). Accordingly the present invention further includes a method for the preparing a plant seed derived product from plants seeds comprising:
(a) providing a chimeric nucleic acid construct comprising in the 5′ to 3′ direction of transcription as operably linked components:
   (i) a nucleic acid sequence capable of controlling expression in plant cells; and
   (ii) a nucleic acid sequence which upon transcription generates a RNA nucleic acid sequence that is complementary to a nucleic acid sequence encoding an oleosin mRNA or a fragment thereof;
(b) introducing the chimeric nucleic acid construct into a plant cell to obtain a transformed plant cell;
(c) regenerating from said transformed plant cell a transformed plant capable of setting seed;
(d) harvesting said seed wherein said seed has a modified oleosin profile; and
(e) preparing a plant seed derived product from said seed
   wherein said seed derived product is a purified protein.

The oil bodies with a suppressed level of endogenous oleosins with concomitant expression of a recombinant oleosin have an increased density or expression level of recombinant oleosins on the surface of said oil bodies when compared to the expression level of a recombinant oleosin in a wild type plant where the endogenous oleosins are not suppressed. In a preferred embodiment, the expression level of the recombinant oleosin on an oil body from a plant with suppressed levels of endogenous oleosins is increased to a level from about 1% to about 20% when compared to the expression level of a recombinant oleosin on an oil body from a plant where the endogenous oleosins are not suppressed. More preferably, the expression level of the recombinant oleosin on an oil body from a plant with suppressed levels of endogenous oleosins is increased to a level from about 10% to 20% when compared to the expression level of a recombinant oleosin on an oil body from a plant where the endogenous oleosins are not suppressed and most preferably, the expression level of the recombinant oleosin on an oil body from a plant with suppressed levels of endogenous oleosins is increased to a level from about 15% to 20% when compared to the expression level of a recombinant oleosin on an oil body from a plant where the endogenous oleosins are not suppressed. Techniques to determine the recombinant oleosin protein levels include densitometry, a quantitative Western blot analysis or the use of an ELISA. Examples of protocols can be found in Coligan et al. Current Protocols in Protein Science, vol 3.

### Use of Oil Bodies with an Increase in the Amount of Recombinant Fusion Proteins on the Surface of Oil Bodies as an Affinity Matrix

The present invention further describes uses of oil bodies with an increased accumulation of recombinant oleosin covalently linked to a second recombinant protein on the surface of oil bodies. In the preferred embodiment, the oil body with an increased accumulation of recombinant fusion proteins in the surface of the oil body can be used as an affinity matrix (see WO 98/27115 and related applications, all which are incorporated herein by reference). As described in WO 98/27115, it was found that oil bodies and their associated proteins can be used as affinity matrices for the separation of a wide variety of target molecules such as proteins, carbohydrates, lipids, organic molecules, nucleic acids, metals, cells and cell fractions from a sample. In accordance with the invention, there is provided a method for the separation of a target molecule from a sample comprising: 1) contacting (i) oil bodies that can associate with the target molecule through a ligand or second recombinant protein which is covalently attached to a recombinant oleosin with (ii) a sample containing the target molecule; and 2) separating the oil bodies associated with the target molecule from the sample. The oil bodies and the sample containing the target molecule are brought into contact in a manner sufficient to allow the oil bodies to associate with the target. Preferably, oil bodies are mixed with the target. If desired, the target molecule may be further separated from the oil bodies. In one example, the ligand fused to the oil body protein may be hirudin and can be used to purify thrombin. In another example, the ligand fused to the oil body protein may be metallothionein and can be used to separate cadmium from a sample. In a further example, the ligand fused to the oil body protein may be protein A and can be used to separate immunoglobulins. In yet another example, the ligand fused to the oil body protein may be cellulose binding protein and can be used to separate cellulose from a sample.

### Use of Oil Bodies with an Increase in the Amount of Recombinant Fusion Proteins on the Surface of Oil Bodies as an Immunogenic Formulation

The present invention further describes uses of oil bodies with an increased accumulation of recombinant oleosin covalently linked to a second recombinant protein on the surface of oil bodies. In a preferred embodiment, the low endogenous oleosin background could allow the display of antigenic polypeptides on the surface of oil bodies, improving their use as an adjuvant in a immunogenic formulation or a vaccine or as an immunogenic formulation (see WO 01/95934 and related applications and U.S. Pat. No. 6,761,914 and related patents and patent applications, all which are incorporated herein by reference). In preferred embodiment, a recombinant oleosin is covalently linked to the antigen or second recombinant protein (as disclosed in WO 93/21320 and related applications which are incorporated by reference in their entirely) which can be physically associated with the oil bodies in the vaccine or immunogenic formulation. The vaccines or immunogenic formulations of the present invention can be used to elicit an immune response against any antigen using any route of administration including transdermal or through the mucosa.

### EXAMPLES

### Example 1

### Construction of Oleosin Suppression Cassettes

Antisense Cassette

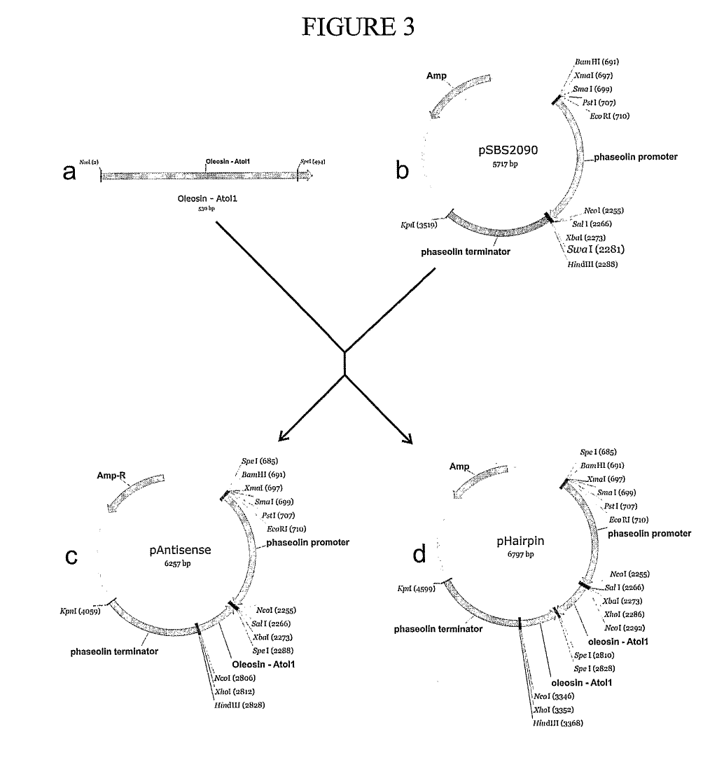

The Atol1 cDNA was amplified using the forward primer NTD (5′-TATTAAGCTTCCATGGCCGATACTGCTAGAGG-3′) (SEQ ID NO:92) containing HindIII and NcoI restriction sites (underlined) and the reverse primer CTR (5′-AGCCATACTAGTAGTGTGTTGACCACCACGAG-3′) (SEQ ID NO:93) containing the SpeI restriction site (underlined) using Atol1 cDNA (SEQ ID NO:94) as a template. The PCR product was purified and inserted in the vector pSBS2090, under control of the phaseolin promoter/terminator (Slightom et al., 1983 Proc. Natl. Acad. Sci. U.S.A. 80:1897-1901.). This vector was previously digested with the restriction enzyme SwaI (FIG. 3b). The PCR product can be inserted in a direct or inverted orientation because the enzyme SwaI generates blunt ends. The plasmids containing Atol1 cDNA in the inverse orientation was screened with the enzyme NcoI. A vector containing Atol1 cDNA in the inverse orientation digested with NcoI releases a DNA fragment with 55 lbp (FIG. 3c).

Hairpin Cassette

The hairpin cassette was constructed following the same scheme described for the antisense. The only difference was that during the ligation reaction the amount of the PCR product was increased to allow the insertion of two inverted repeats of Atol1 cDNA in pSBS2090. The number of copies of Atol1 inserted was analyzed through digestion with Xba and HindIII. Each copy of Atol1 was 555 bp in length and a dimer would have 1110 bp. To check the orientation of the two copies, digestion with NcoI was performed. If the vector contained an inverted repeat of Atol1 cDNA, digestion with NcoI would release a fragment with 1054 bp (FIG. 3d).

Hairpin and Intron Cassette

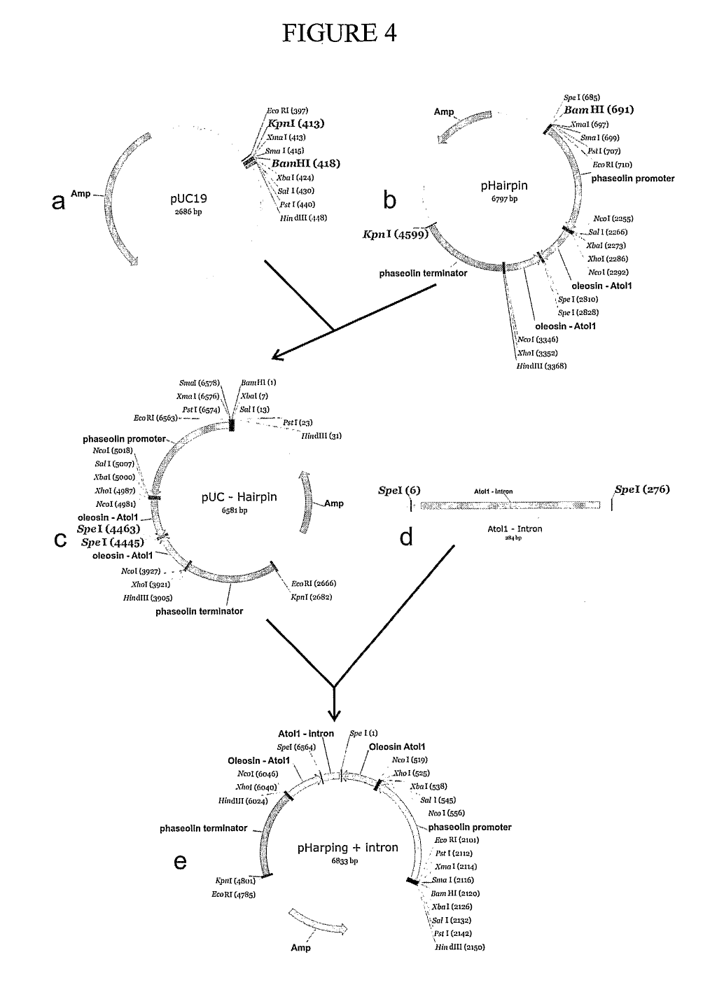

The hairpin+intron cassette was constructed by inserting an intron in the hairpin cassette. The unique intron of Atol1 was amplified using the forward primer IntronD (5′-TTTTACTAGTGATTTACAAtTAAGCACACATTTATC-3′) (SEQ ID NO:95) containing SpeI restriction site (underlined) and the reverse primer IntronR (5′-CTGTACTAGTTCTCCCGTTGCGTACCTATTCAC-3′) (SEQ ID NO:96) containing the SpeI restriction site (underlined) using an Atol1 genomic clone as template. The PCR product was purified and digested with SpeI (FIG. 4d). The hairpin+intron cassette was sub-cloned in the plasmid pUC19 (New England Biolabs Inc.) in the Kpn and BamHI restriction sites (FIG. 4c). The resulting vector was digested with SpeI restriction enzyme between the inverted repeats of Atol1 cDNA. The Atol1 intron was inserted between the repeats (FIG. 4e). The orientation of the insertion was verified through PCR.

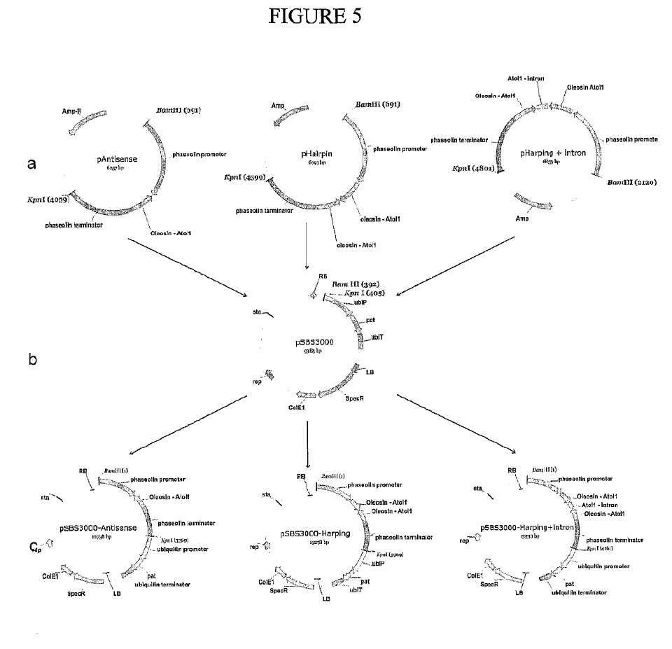

The antisense (SEQ ID NO:97), hairpin (SEQ ID NO:98) and hairpin+intron cassettes (SEQ ID NO:99) are inserted in the binary vector pSBS3000 in the sites KpnI and BamHI (FIG. 5), creating the vectors pSBS3000-antisense, pSBS3000-hairpin and pSBS3000-hairpin+intron.

### Example 2

### Agrobacterium and Arabidopsis Transformation

The binary vectors pSBS3000-antisense, pSBS3000-hairpin and pSBS3000-hairpin+intron were individually inserted in Agrobacterium EHA101 (Hood, E. E. et al. 1986. Journal of Bacteriology 168:1291-1301) by electroporation method. The transformed Agrobacterium lines containing the binary vector were selected using spectinomycin resistance (“SpecR” in FIG. 5e). One line of Agrobacterium was selected for each construct.

Arabidopsis thaliana ecotype C24 was used for transformation. Five seeds were planted on the surface of a soil mixture (two-thirds Redi-earth and one-third perlite with a pH=6.7) in 4 inch pots. The seedlings were allowed to grow to a rosette stage of 6-8 leaves to a diameter of approximately 2.5 cm. These seedlings were transplanted into 4 inch pots containing the above soil mixture, covered with window screen material which has five 1 cm diameter holes cut into the mesh; one in each of the corners, and one in the center. The pots were placed inside a dome at 4° C. for four days for a cold treatment and subsequently moved to 24° C. growth room with constant light at about 150 μE and 50% relative humidity. The plants were irrigated at 2-3 day interval and fertilized weekly with 1% of Peters 20-20-20. When stems reach about 2 cm in height, the primary bolts were cut to encourage the growth of secondary and tertiary bolts. Four to five days after cutting the primary bolts, the plants were ready to be infected with Agrobacterium.

The Agrobacterium lines were individually inoculated in 500 ml of LB media and grown until they reached an optical density of 0.8 at 600 nm. The cultures were centrifuged to precipitate the bacteria that was subsequently suspended in a solution containing 5% of sucrose and 0.05% of the surfactant Silwet L-77 (Lehle Seeds).

The pots with Arabidopsis plants were inverted in the solution for 20 seconds. The pots were subsequently covered with a transparent plastic dome for 24 hours to maintain higher humidity. The plants were allowed to grow to maturity and seeds (untransformed and transformed) were harvested.

For selection of transgeric lines, the putative transformed seeds were sterilized with a quick wash of 70% ethanol and a treatment in 20% commercial bleach for 15 min. The bleach solution was removed by rinsing seeds four times with water. About 1000 sterilized seeds were mixed with 0.6% top agar and evenly spread on a half strength MS plate (Murashige and Skoog 1962. Physiologia Plantarum 15:473-497) containing 1% sucrose and 80 μM of the herbicide phosphinothricin (PPT) DL. The plates were then placed in a growth room with light regime consisting of 8 hours in the dark and 16 hours in the light at 24° C. After 7 to 10 days, putative transgenic seedlings were green and growing whereas untransformed seedlings were dead. After the roots have established the putative transgenic seedlings were individually transferred to pots (the individual plants were irrigated in 3 day intervals and fertilized with 1% Peters 20-20-20 in 5 day intervals and allowed to grow to maturity). The pots were covered with a transparent plastic dome for three days to protect the sensitive seedlings. After 7 days the seedlings were covered with a seed collector from Lehle Seeds to prevent seed loss due to scattering. Seeds from these transgenic plants are harvested individually and ready for analysis.

### Example 3

### Isolation of Oil Bodies

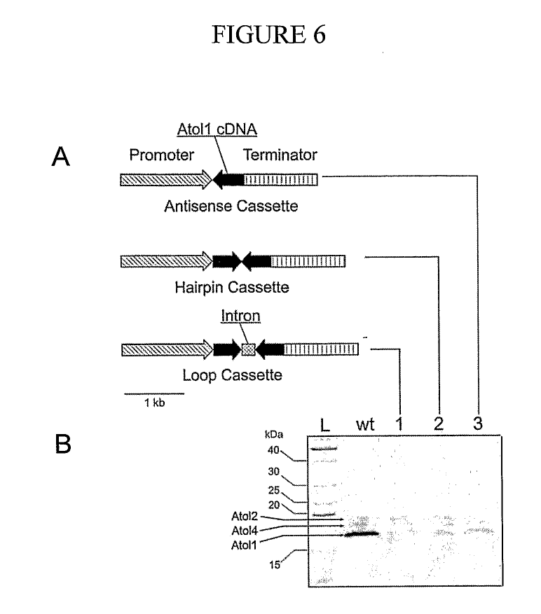

The accumulation of Atol1 in seeds recovered from the selected plants was analyzed by SDS-PAGE of the oil body fraction. Oil bodies from these seeds were obtained using the method reported by van Rooijen & Moloney, (1995) Biotechnology (N.Y.) 13, 72-77 with the following modifications. Briefly, 10 to 20 mg of dry mature seeds were ground inside a 1.7 ml microfuge tube with 0.4 ml of oil body extraction buffer (50 mM Tris-HCl pH 7.5 with 0.4M of sucrose and 0.5M of NaCl). The extract was centrifuged for 15 min at 10,000 g at room temperature (RT). After centrifugation the fat pad containing the oil bodies was removed from the aqueous phase and transferred to another microfuge tube. The oil bodies were resuspended in 0.4 ml of high stringency urea buffer (8M Urea in 100 mM Na-Carbonate buffer pH 8.0). The sample was centrifuged for 15 min at 10,000 g at 4° C. and the undernatant removed. The oil bodies were finally suspended in 0.1 ml of water. The presence of Atol1 in oil body fractions was detected by loading 20 μl of oil body fraction in SDS-PAGE 15% and staining with Coomassie blue (FIG. 6). A decrease in the level of Atol1 was observed in the oil body fraction for all of the cassettes (Antisense—lane 3, Hairpin—lane 2 and Hairpin-Loop—lane 1) with a higher level of suppression in the Hairpin-Loop cassette when compared to the wild type oil body fraction.

### Example 4

### Microscopy Analysis of Oil Bodies

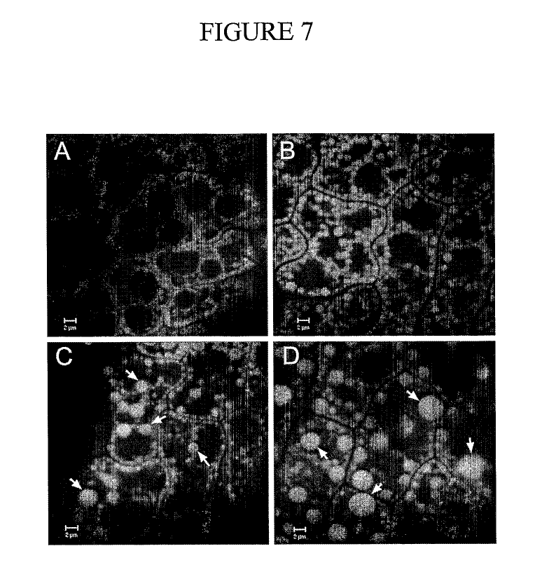

Morphological analysis of oil bodies can be performed in vivo using dark field confocal microscopy or in vitro using bright field conventional microscopy. Mature embryos were isolated according the method established by Perry and Wang (2003 Biotechniques 35:278-281). For confocal microscopy (dark field microscopy) isolated embryos were infiltrated with an aqueous solution of 10 μg/ml Nile red (Molecular Probes) for neutral lipid staining (Greenspan et al., 1985. J. Cell Biol. 100:965-973). Stained embryos were examined with a Zewass LSM 510 laser scanning confocal microscope using line-sequential single-tracking mode with the AOTF-controlled excitation with 488 nm and 543 mm laser (set at 20% and 100% respectively). A Plan-Appochromat 40×/1.4 Oil DIC objective was used with scan zoom. The pinhole was optimized for about 100 μm. The resulting micrographs are shown in FIG. 7. In wild type embryos oil bodies are present in the boundaries of the cells as small units with about 1 μm (FIG. 7A). In embryos obtained from transgenic plants transformed with pSBS3000-antisense, pSBS3000-hairpin and pSBS3000-hairpin+intron oil bodies were considerably larger, being up to 6 μm in diameter. (FIGS. 7B, 7C and 7D, respectively). Although the oil bodies from transgenic plants transformed with pSBS3000-antisense remained relatively uniform in size, oil bodies from the plants transformed with hairpin and hairpin+intron were very heterogenous in size. The size of these oil bodies ranged from that similar to the wild type to several times larger.

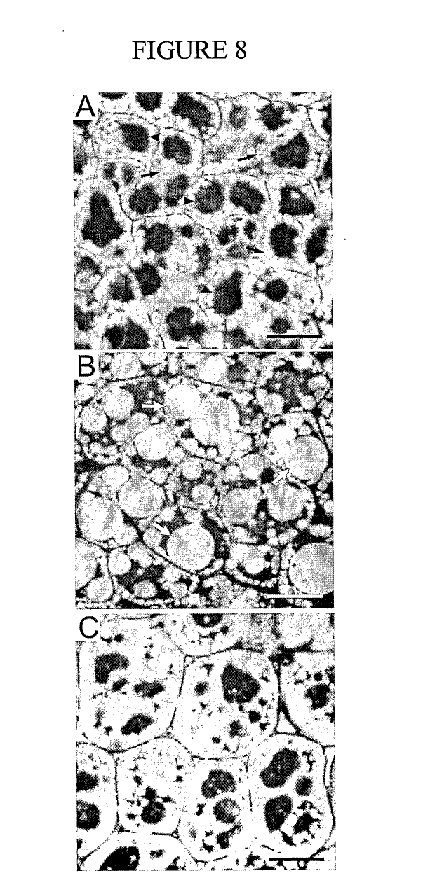

For bright field microscopy isolated embryos were fixed immediately in 2.5% glutaraldehyde and 1.6% paraformaldehyde in a 0.1M phosphate buffer, pH 6.8 for 4 hours. After rinsing several times in the same buffer, the embryos were post-fixed with a 2% osmium tetroxide solution for an additional 4 hours. The osmium tetroxide solution was used for lipid fixation in the specimens. After dehydration using the acetone series, the embryos were infiltrated and subsequently embedded in the Ladd LX-112 epoxy resin. Semi-thin sections are obtained using a Sorval MT-1 ultramicrotome. The sections were stained using periodic acid-Schiff's reaction and counterstained with an alkaline toluidine blue O solution (Yeung, 1990. Stain Technol. 65:45-47.). The resulting micrographs show the embryo cell structure where the protein bodies are stained in purple and the oil bodies are present as hollow structures (FIG. 8). In wild type embryos oil bodies were present as small units (1 μm) (FIG. 8A). As demonstrated for confocal microscopy, the oil bodies obtained from transgenic plants transformed with pSBS3000-hairpin+intron and pSBS3000-hairpin were considerably more heterogeneous in size ranging from sizes similar to that of the wild type to large (up to 6 μm) and protein bodies were very irregular (FIG. 8B). Again the oil bodies from transgenic plants transformed with pSBS3000-antisense were large, but uniform. Additionally, a wild type like (null) Arabidopsis line lacking the pSBS3000-hairpin+intron cassette was propagated from the pSBS3000-hairpin+intron transformed parent line. The oil bodies from this null line displayed a phenotype more similar to that of the wild type than that of the parental transgenic plant (FIG. 8C).

### Example 5

### Measurement of Lipid, Protein, Sucrose and Starch Content

Seed lipid accumulation in plants transformed with Hairpin+intron cassette was analyzed according to the method described by Bligh and Dyer (1959. Can. J. Med. Sci. 37:911-917) with modifications. Fifty milligrams of seeds were homogenized in liquid nitrogen and incubated at 70° C. for 10 minutes with 5 ml of isopropanol. The isopropanol was evaporated and lipids were extracted with three extractions of chloroform, methanol and water biphasic solutions (MeOH:CHCl₃:H₂O). The first extraction was performed with 5.8 ml of MeOH:CHCl₃:H₂O (2:2:1.8 [v/v]) and the second and third extractions are performed with 2.0 ml of MeOH:CHCl₃:H₂O (1:2:0.8 [v/v]). The lipid fractions were collected and the solvents were evaporated under a nitrogen environment. Total lipids were quantified by gravimetric analysis.

Seed protein accumulation in plants transformed with Hairpin+intron cassette was analyzed using the BCA protein assay reagent (Pierce, Rockford, Ill.). Total seed proteins were extracted from 50 mg of seeds homogenized in 1.5 ml of protein extraction buffer (2% SDS, 5 mM EDTA, 50 mM Tris-HCl, pH 6.8). The homogenates were placed in boiling water for 5 minutes and centrifuged at full speed for 10 minutes. The upper phase was removed and the debris was washed two times with 0.5 ml of extraction buffer. The fractions were pooled and the amount of protein was measured with the BCA protein assay reagent.

The analysis of carbohydrates in plant transformed with the Hairpin+intron cassette was performed as described by Focks and Benning (1998) Plant Phys. 118:91-101.) with some modifications. Five milligrams of seeds were homogenized in 0.5 ml of 80% (v/v) ethanol and incubated at 70° C. for 90 minutes. The homogenate was centrifuged at full speed for 5 minutes and the supernatant was transferred to a new test tube. The pellet was washed three times with 0.5 ml of 80% ethanol and the solvent of the combined supernatants was evaporated at room temperature under vacuum. The residue, which represents the soluble carbohydrate fraction, was dissolved in 0.1 ml of water and used for sucrose quantification. The insoluble fraction from the ethanol extraction was suspended in 0.2 ml of 0.2M KOH and incubated 95° C. for 1 h. The solution was neutralized with 35 ml of 1M acetic acid and centrifuged for 5 minutes at full speed. The supernatant was used for starch quantification. Sucrose and starch were determined using kits from Sigma-Aldrich (Oakville, ON).

The suppression of Atol1 isoform of oleosin resulted in a decrease in lipid accumulation accompanied by an increase in protein content. While the ratio of lipid and protein content changed, the total weight of protein and oil remained constant. Analysis of sucrose and starch content revealed no significant difference in the accumulation of these carbohydrates. (Table 2).

### Example 6

### Effect on Oil Body Composition

Lipid and protein accumulation in the oil bodies of wild type plants and plants transformed with Hairpin+intron cassette were analyzed. Oil bodies were isolated from seeds and suspended in water. Total lipids were extracted from aliquots oil body suspension with methanol and chloroform through the method described by Bligh and Dyer (1959). Three extractions were performed and the solvents were evaporated under nitrogen environment. Total lipids were quantified through gravimetry. To measure the amount of proteins, the oil body suspension was boiled in the presence of 2% of SDS and centrifuged. The undernatant, containing the oil body proteins, was collected and used in BCA protein assays. The percentage of lipids and proteins was calculated considering the sum of both masses as the total mass of oil bodies. The oil bodies from transgenic plants transformed with pSBS3000-hairpin+intron cassette contained less protein, or a lower oleosin-to-TAG ratio, than those from the wild type plants.

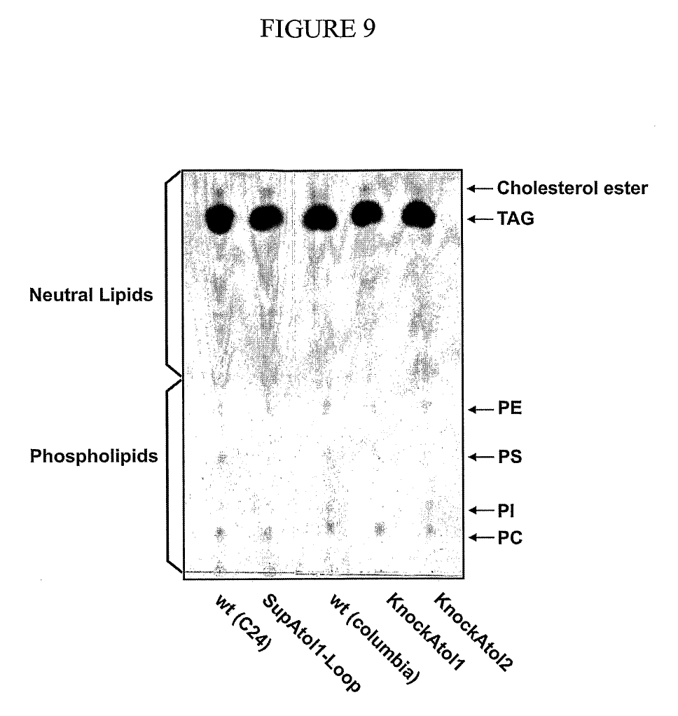

The composition of lipids in the oil body fraction was evaluated through thin layer chromatography. Total lipids extracted from oil bodies were loaded in similar amounts on a silica-gel plate. The plate was half-developed with the mixture chloroform-methanol-acetic acid-formic acid-water (70:30:12:4:2 [v/v]) to resolve phospholipids and fully developed with the mixture hexane-diethyl ether-acetic acid (65:35:2 [v/v]) to allow the separation of neutral lipids (Vance and Russel, 1990). Lipids were visualized through charring in the presence of cupric sulphate. The majority of lipid was composed of TAG with smaller amounts of cholesterol ester and other neutral lipids. For the phospholipids, the most abundant was phosphatidylcholine (PC), followed by phosphatidylethanolamine (PE), phosphatidylserine (PE) and phosphatidylinositol (PI). Oil bodies from pSBS3000-hairpin+loop transformed plants exhibited a slight reduction in phospholipids content, especially phosphatidylcholine and phosphatidylinositol, as compared to oil bodies from wild type plants (FIG. 9).

### Example 7

### Reversion to Wild-Type Phenotype

To explore the possibility of restoring the phenotype by increasing the amount of oleosins, a gene coding for a recombinant oleosin was introduced in the Hairpin-Loop line. To avoid cross-suppression through PTGS, an oleosin from Maize (MaizeOle1, accession number U13701) was selected to restore the function because it is phylogenetically distant from Atol1 (Huang, 1996; Lee et al., 1994).

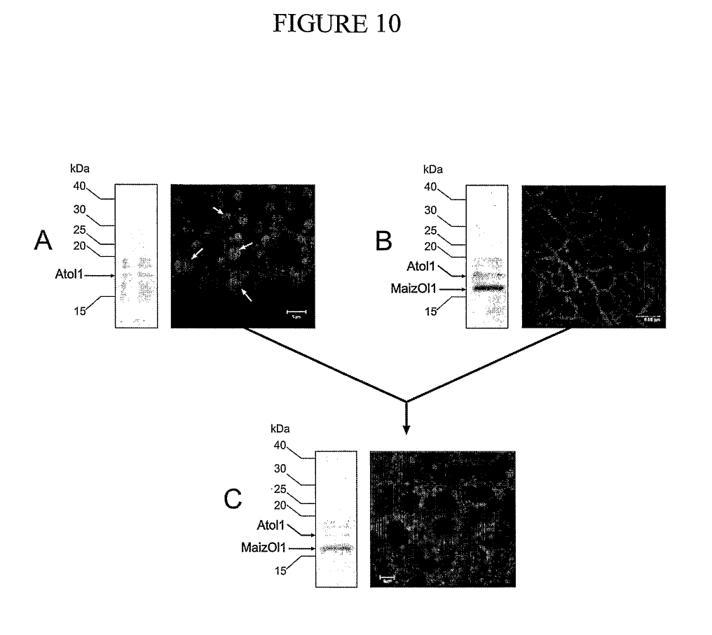

The Hairpin-Loop Arabidopsis plant (FIG. 10A) was manually crossed with MaizOl, an Arabidopsis line expressing MaizeOle1 under control of the linin seed specific promoter (FIG. 10B). MaizeOle1 has distinct molecular weight (15.8 kDa) when compared to Arabidopsis oleosins. We used this property to analyze the progeny of the crossed lines. The lines showing the presence of Maize oleosin and suppression of Atol1 were selected and propagated for two more generations. A homozygous line was obtained and the seeds were analyzed in a confocal microscope (FIG. 10C). Oil bodies in this line did not display the phenotype found in the Hairpin-Loop line. Such oil bodies were still larger than wild-type ones but uniform in size, like those found in the Antisense line. This result indicates that the size of the oil bodies is controlled by the level of oleosin protein.

### Example 8

### Effect on Germination and Fate of Oil Bodies During Seedling Development

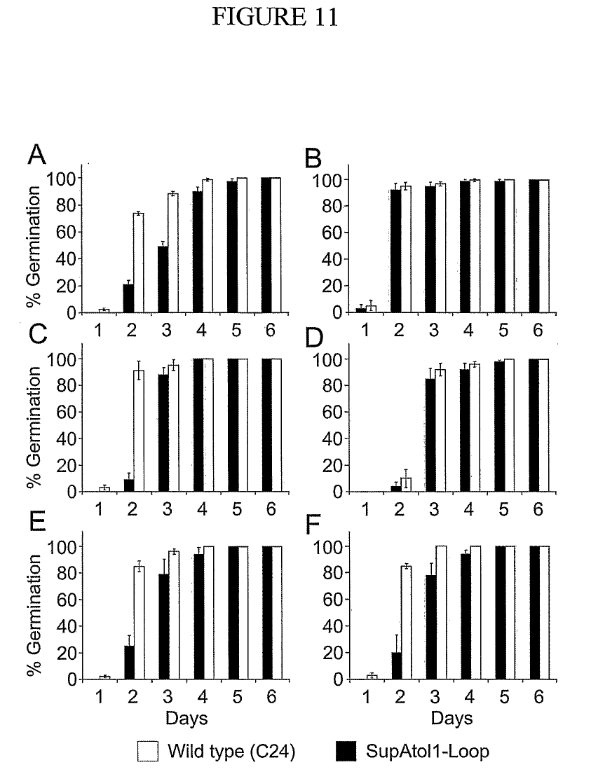

Microscopy analysis clearly showed that suppression of oleosins resulted in alteration of oil body production and affected the organization of protein storage organelles. Since both TAGs and storage proteins are used for seedling development, the effects of this aberrant sub-cellular morphology during seedling growth were investigated. Germination tests were conducted in different conditions of carbohydrate availability and light exposure and noted a delay in germination of SupAtol1-Loop (Hairpin-Loop) compared with wild-type seeds (FIG. 11). The most prominent difference was found during the second and third days for seeds germinated on moistened paper (FIG. 11A). No significant difference was found for seeds germinated under light exposure with sucrose supplement (FIG. 11B).

When the Hairpin-Loop line was stratified for 3 days and subsequently germinated in the light, the delay in germination that was observed above was masked (FIG. 11B). However, when the Hairpin-Loop line was stratified for 3 days and subsequently germinated in the dark, the delay in germination was an intermediate between no stratification and stratification with germination in the light.

When seeds were germinated in MS media with or without sucrose kept in the dark (FIGS. 11E and 11F) or media without sucrose exposed to light (FIG. 11C). The delay in seed germination could be reverted when seeds were sown in media with sucrose and exposed to light (FIG. 11D) or when seeds were sown on moistened paper and submitted to stratification for 3 days (FIG. 11B).

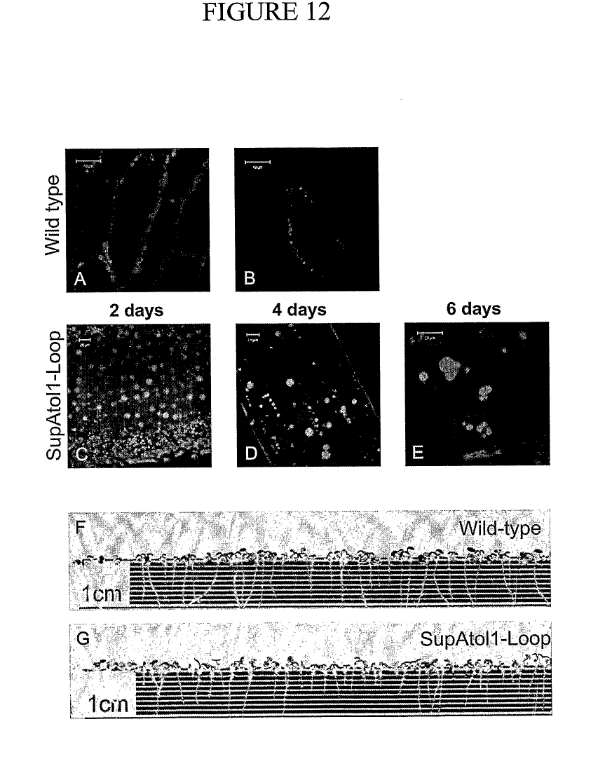

After germination the development of oleosin-suppressed seedlings was comparable to wild type (FIGS. 12F and G). Usually oil bodies are consumed during the first days after imbibition. Our experiments demonstrated that, two days after imbibition, the oil bodies were still present in the boundaries of the cells as small units. After four days they are scarcely found and have completely disappeared at the fifth day (FIGS. 12A and 12B). In oleosin suppressed plants oil bodies assume different behaviour. Two days after germination they are found as large structures with about 10 μm in the cytoplasm. Four days after imbibition the number and size and of oil bodies decrease although large structures are still present in some cells. Six days after germination some oil bodies can still be found as large structures but after that they completely disappear (FIGS. 12C to 12E). The slower mobilization of TAGs does not seem to affect post germination growth (FIGS. 12F and 12G). Although some seedlings seem to be smaller in the Hairpin-Loop line this is most likely due to a delay in germination.

The present invention should therefore not be seen as limited to the particular embodiments described herein, but rather, it should be understood that the present invention has wide applicability with respect to protein expression generally. Since modifications will be apparent to those of skill in this art, it is intended that this invention be limited only by the scope of the appended claims.

### SUMMARY OF SEQUENCES

SEQ ID NO:1 to 84 set forth known oleosin sequences which are described in Table 1.

SEQ ID NO:85 and 86 set forth the nucleic acid sequences of the antisense sequences.

SEQ ID NO:87 and 88 set forth nucleic acid sequences of the sense sequences.

SEQ ID NO:89 to 91 set forth the nucleic acid sequences of the loop sequences.

SEQ ID NO:92 sets forth the nucleotide sequence of the forward primer NTD which is complementary to the 5′ region of the Atol 1 cDNA clone and is designed to add HindIII and NcoI restriction sites site to the 5′ region facilitate subsequent ligation.

SEQ ID NO:93 sets forth the nucleotide sequence of the reverse primer CTR which is complementary to 3′ region of the C-terminal domain of Atol 1 cDNA and is designed to add a SpeI site to the 3′ region facilitate subsequent ligation.

SEQ ID NO:94 sets forth the nucleotide sequence of the Atol 1 cDNA sequence.

SEQ ID NO:95 sets forth the nucleotide sequence of the forward primer Intron D which is complementary to the 5′ region of the 5′ border of the intron of Atol 1 (including the 3′ region of exon 1) and is designed to add SpeI restriction sites site to the 5′ region facilitate subsequent ligation.

SEQ ID NO:96 sets forth the nucleotide sequence of the reverse primer Intron R which is complementary to the 3′ region of the 3′ border of the intron of Atol 1 (including the 5′ region of exon 2) and is designed to add SpeI restriction sites site to the 3′ region facilitate subsequent ligation.

SEQ ID NO:97 sets forth the nucleotide sequence of the antisense cassette as described in Example 1.

SEQ ID NO:98 sets forth the nucleotide sequence of the hairpin construct as described in Example 1.

SEQ ID NO:99 sets forth the nucleotide sequence of the hairpin and intron cassette as described in Example 1.

TABLE 1
Examples of known oleosin sequences

| | |
|---|---|
| SEQ. ID | Oleosin Motif |
| NO. | (Amino Acid Sequence Identifier) {Nucleic Acid Sequence Identifier} |

Arabidopsis

| | |
|---|---|
| 1 | (A84654) Arabidopsis thaliana probable oleosin |
| 2 | (AAA87295) Arabidopsis thaliana oleosin {Gene L40954} |
| 3 | (AAC42242) Arabidopsis thaliana oleosin {Gene AC005395} |
| 4 | (AAF01542) Arabidopsis thaliana putative oleosin {Gene AC009325} |
| 5 | (AAF69712) Arabidopsis thaliana F27J15.22 {Gene AC016041} |
| 6 | (AAK96731) Arabidopsis thaliana oleosin-like protein {Gene AY054540} |
| 7 | (AAL14385) Arabidopsis thaliana AT5g40420/MPO12_130 oleosin isoform |
| | {Gene AY057590} |
| 8 | (AAL24418) Arabidopsis thaliana putative oleosin {Gene AY059936} |
| 9 | (AAL47366) Arabidopsis thaliana oleosin-like protein {Gene AY064657} |
| 10 | (AAM10217) Arabidopsis thaliana putative oleosin {Gene AY081655} |
| 11 | (AAM47319) Arabidopsis thaliana AT5g40420/MPO12_130 oleosin isoform |
| | {Gene AY113011} |
| 12 | (AAM63098) Arabidopsis thaliana oleosin isoform {Gene AY085886} |
| 13 | (AAO22633) Arabidopsis thaliana putative oleosin {Gene BT002813} |
| 14 | (AAO22794) Arabidopsis thaliana putative oleosin protein {Gene BT002985} |
| 15 | (AAO42120) Arabidopsis thaliana putative oleosin {Gene BT004094} |
| 16 | (AAO50491) Arabidopsis thaliana putative oleosin {Gene BT004958} |
| 17 | (AAO63989) Arabidopsis thaliana putative oleosin {Gene BT005569} |
| 18 | (AAQ22658) Arabidopsis thaliana At4g25140 {Gene BT010189.1} |
| 19 | (AAQ56108) Arabidopsis lyrata subsp. Lyrata Oleosin. {Gene AY292860} |
| 20 | (BAA97384) Arabidopsis thaliana oleosin-like {Gene AB023044} |
| 21 | (BAB02690) Arabidopsis thaliana oleosin-like protein {Gene AB018114} |
| 22 | (BAB11599) Arabidopsis thaliana oleosin, isoform 21K {Gene AB006702} |
| 23 | (BAC42839) Arabidopsis thaliana putative oleosin protein {Gene AK118217} |
| 24 | (BAD94320) Arabidopsis thaliana oleosin {Gene AK220898.1} |
| 25 | (CAA44225) Arabidopsis thaliana oleosin {Gene X62353} |
| 26 | (CAA63011) Arabidopsis thaliana oleosin, type 4 {Gene X91918} |
| 27 | (CAA63022) Arabidopsis thaliana oleosin, type 2 {Gene X91956} |
| 28 | (CAA90877) Arabidopsis thaliana oleosin {Gene Z54164} |
| 29 | (CAA90878) Arabidopsis thaliana oleosin {Gene Z54165} |
| 30 | (CAB36756) Arabidopsis thaliana oleosin, 18.5 K {Gene AL035523} |
| 31 | (CAB79423) Arabidopsis thaliana oleosin, 18.5 K {Gene AL161562} |
| 32 | (CAB87945) Arabidopsis thaliana oleosin-like protein {Gene AL163912} |
| 33 | (P29525) Arabidopsis thaliana oleosin 18.5 kDa {Gene X62353, CAA44225, |
| | AL035523, CAB36756, CAB36756, CAB79423, Z17738, S22538} |
| 34 | (Q39165) Arabidopsis thaliana Oleosin 21.2 kDa (Oleosin type 2). {Gene |
| | L40954, AAA87295, X91956, CAA63022, Z17657, AB006702, BAB11599, |
| | AY057590, AAL14385, S71253 |
| 35 | (Q42431) Arabidopsis thaliana Oleosin 20.3 kDa (Oleosin type 4) {Gene |
| | Z54164, CAA90877, X91918, CAA63011, AB018114, BAB02690, AY054540, |
| | AAK96731, AY064657, AAL47366, AY085886, AAM63098, Z27260, |
| | Z29859, S71286 |
| 36 | (Q43284) Arabidopsis thaliana Oleosin 14.9 kDa. {Gene Z54165, CAA90878, |
| | AB023044, BAA97384, Z27008, CAA81561} |
| 37 | (S22538) Arabidopsis thaliana oleosin, 18.5 K |
| 38 | (S71253) Arabidopsis thaliana oleosin, 21 K |
| 39 | (S71286) Arabidopsis thaliana oleosin, 20 K |
| 40 | (T49895) Arabidopsis thaliana oleosin-like protein |

Brassica

| | |
|---|---|
| 41 | (AAB22218) Brassica napus oleosin napII |
| 42 | (AAB22219) Brassica napus oleosin napI |
| 43 | (AAD24547) Brassica oleracea oleosin |
| 44 | (AAK38471) Brassica oleracea putative oleosin {Gene AY028608.1} |
| 45 | (AAK38472) Brassica oleracea putative oleosin {Gene AY028608.1} |
| 46 | (AAK38473) Brassica oleracea putative oleosin {Gene AY028608.1} |
| 47 | (AAK38474) Brassica oleracea putative oleosin {Gene AY028608.1} |
| 48 | (AAK38475) Brassica oleracea putative oleosin {Gene AY028608.1} |
| 49 | (AAW70038) Brassica rapa oleosin-like protein {Gene AY747625.1} |
| 50 | (CAA41064) Brassica napus oleosin Nap-II {Gene X58000.1} |
| 51 | (CAA43941) Brassica napus oleosin BN-III {Gene X63779} |
| 52 | (CAA45313) Brassica napus oleosin BN-V {Gene X63779} |
| 53 | (CAA57544) Brassica napus oleosin {Gene X82019.1} |
| 54 | (CAA57545) Brassica napus oleosin {Gene X82020.1} |
| 55 | (CAA64800) Brassica napus oleosin-like protein {Gene X95554.1} |
| 56 | (CAA64801) Brassica napus oleosin-like protein {Gene X95555.1} |
| 57 | (CAA64802) Brassica napus oleosin-like protein {Gene X95556.1} |
| 58 | (CAA64803) Brassica napus oleosin-like protein {Gene X95557.1} |
| 59 | (CAA64804) Brassica napus oleosin-like protein {Gene X95558.1} |
| 60 | (CAA64805) Brassica napus oleosin-like protein {Gene X95559.1} |
| 61 | (CAA64806) Brassica napus oleosin-like protein {Gene X95560.1} |
| 62 | (CAA70173) Brassica napus oleosin-like protein {Gene Y08986.1} |
| 63 | (P29109) Brassica napus Oleosin Bn-V (BnV) {Gene X63779, CAA45313, |
| | S25089) |
| 64 | (P29110) Brassica napus Oleosin Bn-III (BnIII) {Gene X61937, CAA43941, |
| | S22475) |
| 65 | (P29111) Brassica napus Major oleosin NAP-II {Gene X58000, CAA41064, |
| | S70915) |
| 66 | (P29526) Brassica napus oleosin C98 {Gene X67142.1, CAA47623.1, S24960} |
| 67 | (S13494) Brassica napus Major Oleaosin Chain Nap-I - rape (fragment) |
| 68 | (S22475) Brassica napus oleosin BN-III |
| 69 | (S25089) Brassica napus oleosin BN-V - rape (fragment) |
| 70 | (S50195) Brassica napus Oleosin |
| 71 | (S70915) Brassica napus Major Oleosin NAP-II - rape (fragment) |
| 72 | (T08134) Brassica napus Oleosin-like |
| 73 | (1803528A) Brassica napus Oleosin |
| 74 | (2009397A) Brassica napus Oleosin |

Daucus carota (carrot)

| | |
|---|---|
| 75 | (AAB01098) Daucus carota oleosin |
| 76 | (T14307) carrot oleosin |

Maize

| | |
|---|---|
| 77 | (A35040) Zea mays oleosin 18 |
| 78 | (AAA67699) Zea mays oleosin KD18 {Gene J05212} |
| 79 | (AAA68065) Zea mays 16 kDa oleosin {Gene U13701} |
| 80 | (AAA68066) Zea mays 17 kDa oleosin {Gene U13702} |
| 81 | (P13436) Zea mays OLEOSIN ZM-I (OLEOSIN 16 KD) (LIPID BODY- |
| | ASSOCIATED MAJOR PROTEIN) {Gene U13701, AAA68065, M17225, |
| | AAA33481, A29788} |
| 82 | (P21641) Zea mays Oleosin Zm-II (Oleosin 18 kDa) (Lipid body-associated |
| | protein L2) {Gene J05212, AAA67699, A35040} |
| 83 | (S52029) Zea mays oleosin 16 |
| 84 | (S52030) Zea mays oleosin 17 |

TABLE 2
Lipid, Protein and Carbohydrate Content in Wild-Type and
Hairpin-Loop Seeds

| | Total lipid | n | Total Protein | n |
|---|---|---|---|---|
| Wild-type (C24) | 40.25% ± 1.36 | 5 | 25.09% ± 1.71 | 8 |
| Hairpin-Loop | 32.91% ± 2.00 | 5 | 33.87% ± 1.61 | 8 |
| | Total Starch | n | Total Sucrose | n |
| Wild-type (C24) | 0.5% ± 0.3 | 5 | 3.2% ± 0.4 | 5 |
| Hairpin-Loop | 0.8% ± 0.4 | 5 | 2.8% ± 0.2 | 5 |

TABLE 3
Lipid and Protein Content in Wild-Type and Hairpin-Loop Oil
Bodies

| | Total lipid | Total Protein |
|---|---|---|
| Wild-type (C24) | 98.0% | 2.0% |
| Hairpin-Loop | 99.1% | 0.9% |

1: A method for preparing a plant seed derived product from plant seeds comprising:
a) providing a chimeric nucleic acid construct comprising in the 5′ to 3′ direction of transcription as operably linked components:
(i) a nucleic acid sequence capable of controlling expression in plant seed cells; and
(ii) a nucleic acid sequence which upon transcription generates a RNA nucleic acid sequence that is complementary to a nucleic acid sequence encoding an oleosin mRNA sequence or fragment thereof;
b) introducing the chimeric nucleic acid construct into a plant cell;
c) regenerating from said transformed plant cell a transformed plant capable of setting seed;
d) harvesting said seed wherein said seed has a modified oleosin profile; and
e) preparing a plant seed derived product from said seed.

2: A method according to claim 1 wherein said nucleic acid sequence (ii) comprises SEQ ID NO:85 or 86.

3: A method according to claim 1 wherein said chimeric nucleic acid construct further comprises (iii) a nucleic acid sequence encoding an oleosin or a fragment thereof wherein said nucleic acid sequence is complementary to the nucleic acid sequence provided in (ii).

4: A method according to claim 3 wherein said nucleic acid sequence (iii) encoding an oleosin or a fragment thereof comprises SEQ ID NO:87 or 88.

5: A method according to claim 3 wherein said nucleic acid sequence (ii) and said nucleic acid sequence (iii) are the same length.

6: A method according to claim 5 wherein said chimeric nucleic acid construct forms a hairpin structure.

7: A method according to claim 3 wherein said chimeric nucleic acid construct further comprises a polynucleotide loop structure.

8: A method according to claim 7 wherein said polynucleotide loop structure comprises an oleosin gene intron.

9: A method according to claim 8 wherein said polynucleotide loop structure comprises SEQ ID NO:89, 90 or 91.

10: A method according to claim 1 wherein said seed having a modified oleosin profile has an increase in total protein content and a decrease in lipid content as compared to non-transformed seed.

11: A method according to claim 1 wherein said plant seed is monocotyledonous.

12: A method according to claim 1 where said plant seed is dicotyledonous.

13: A method according to claim 1 wherein said plant seed is selected from the group consisting of rapeseed (Brassica spp.), linseed/flax (Linum usitatissimum), safflower (Carthamus tinctorius), sunflower (Helianthus annuus), maize (Zea mays), soybean (Glycine max), mustard (Brassica spp. and Sinapis alba), crambe, (Crambe abyssinica), eruca (Eruca sativa), oil palm (Elaeis guineeis), cottonseed (Gossypium spp.), groundnut (Arachis hypogaea), coconut (Cocus nucifera), castor bean (Ricinus communis), coriander (Coriandrum sativum), squash, (Cucurbita maxima), Brazil nut (Bertholletia excelsa) and jojoba (Simmondsia chinensis).

14: A method according to claim 1 wherein said plant seed derived product is a food or feed product.

15: A method according to claim 1 wherein said plant seed derived product is an oil body comprising a modified oleosin profile.

16: A method according to claim 15 wherein said oil body comprising a modified oleosin profile is formulated into a personal care product.

17: A method according to claim 1 wherein the chimeric nucleic acid construct is introduced into a plant cell under nuclear genomic integration conditions.

18: A chimeric nucleic acid sequence, capable of being expressed in a plant cell comprising:
(a) nucleic acid sequence capable of regulating the transcription in said plant cell;
(b) a nucleic acid sequence which upon transcription generates a RNA nucleic acid sequence that is complementary to a nucleic acid sequence encoding an oleosin mRNA or a fragment thereof; and
(c) a nucleic acid sequence encoding a termination region functional in the plant cell.

19: A chimeric nucleic acid sequence according to claim 18 wherein said chimeric nucleic acid construct further comprises a nucleic acid sequence encoding an oleosin or a fragment thereof wherein said nucleic acid sequence is complementary to the nucleic acid sequence provided in (b).

20: A chimeric nucleic acid sequence according to claim 19 wherein said nucleic acid sequence encoding an oleosin or a fragment thereof comprises SEQ ID NO:87 or 88.

21: A chimeric nucleic acid sequence according to claim 18 wherein said nucleic acid sequence (b) and said nucleic acid sequence encoding an oleosin or a fragment thereof are the same length.

22: A chimeric nucleic acid sequence according to claim 18 wherein said chimeric nucleic acid construct forms a hairpin structure.

23: A chimeric nucleic acid sequence according to claim 18 wherein said chimeric nucleic acid construct further comprises a polynucleotide loop structure.

24: A chimeric nucleic acid sequence according to claim 23 wherein said polynucleotide loop structure comprises an oleosin gene intron.

25: A chimeric nucleic acid sequence according to claim 24 wherein said polynucleotide loop structure comprises SEQ ID NO:89, 90 or 91.

26: An expression vector comprising said chimeric nucleic acid sequence according to claim 17.

27: A plant transformed with said chimeric nucleic acid sequence according to claim 17.

28: A plant according to claim 27 wherein said plant is monocotyledonous.

29: A plant according to claim 27 where said plant is dicotyledonous.

30: A plant according to claim 27 wherein said plant is selected from the group consisting of rapeseed (Brassica spp.), linseed/flax (Linum usitatissimum), safflower (Carthamus tinctorius), sunflower (Helianthus annuus), maize (Zea mays), soybean (Glycine max), mustard (Brassica spp. and Sinapis alba), crambe, (Crambe abyssinica), eruca (Eruca sativa), oil palm (Elaeis guineeis), cottonseed (Gossypium spp.), groundnut (Arachis hypogaea), coconut (Cocus nucifera), castor bean (Ricinus communis), coriander (Coriandrum sativum), squash, (Cucurbita maxima), Brazil nut (Bertholletia excelsa) and jojoba (Simmondsia chinensis).

31: A composition comprising oil bodies isolated from plant seeds with a modified oleosin profile wherein said oil bodies are at least 2 times larger than wild type oil bodies.

32: A composition according to claim 31 that is prepared by a process comprising:
a) providing a chimeric nucleic acid construct comprising in the 5′ to 3′ direction of transcription as operably linked components:
(i) a nucleic acid sequence capable of controlling expression in plant seed cells; and
(ii) a nucleic acid which upon transcription generates a RNA nucleic acid sequence that is complementary to a nucleic acid sequence encoding an oleosin mRNA or fragment thereof;
b) introducing the chimeric nucleic acid construct into a plant cell;
c) regenerating from said transformed plant cell a transformed plant capable of setting seed; and
d) harvesting said seed and isolating oil bodies from said seed with a modified oleosin profile.

33: A composition according to claims 31 wherein said nucleic acid sequence (ii) comprises SEQ ID NO:85 or 86.

34: A composition according to claim 31 wherein said seed having a modified oleosin profile has an increase in total protein content and a decrease in lipid content as compared to non-transformed seed.

35: A composition according to claim 31 wherein said chimeric nucleic acid construct further comprises a (iii) nucleic acid sequence encoding an oleosin or a fragment thereof wherein said nucleic acid sequence is complementary to the nucleic acid sequence provided in (ii).

36: A composition comprising according to claim 35 wherein said nucleic acid sequence (iii) capable of encoding an oleosin or a fragment thereof is SEQ ID NO:87 or 88.

37: A composition according to claim 35 wherein said nucleic acid sequence (ii) and said nucleic acid sequence (iii) are the same length.

38: A composition according to claim 35 wherein said chimeric nucleic acid construct forms a hairpin structure.

39: A composition according to claim 35 wherein said chimeric nucleic acid construct further comprises a polynucleotide loop structure.

40: A composition comprising oil bodies with a modified oleosin profile according to claim 39 wherein said polynucleotide loop structure consists of an oleosin gene intron.

41: A composition according to claim 40 wherein said polynucleotide loop structure is SEQ ID NO:89, 90 or 91.

42: A composition comprising oil bodies with a modified oleosin profile according to claim 31 wherein said plant seed is monocotyledonous.

43: A composition according to claim 31 where said plant seed is dicotyledonous.

44: A composition according to claim 31 wherein said plant seed is selected from a group comprising is from the species rapeseed (Brassica spp.), linseed/flax (Linum usitatissimum), safflower (Carthamus tinctorius), sunflower (Helianthus annuus), maize (Zea mays), soybean (Glycine max), mustard (Brassica spp. and Sinapis alba), crambe, (Crambe abyssinica), eruca (Eruca sativa), oil palm (Elaeis guineeis), cottonseed (Gossypium spp.), groundnut (Arachis hypogaea), coconut (Cocus nucifera), castor bean (Ricinus communis), coriander (Coriandrum sativum), squash, (Cucurbita maxima), Brazil nut (Bertholletia excelsa) and jojoba (Simmondsia chinensis).
